(19) United States
(12) Patent Application Publication
(10) Pub. No.: US 2020/0011883 A1
(43) Pub. Date: Jan. 9, 2020

(54) METHODS FOR ASSESSING THE PROBABILITY OF ACHIEVING ONGOING PREGNANCY AND INFORMING TREATMENT THEREFROM

(71) Applicant: Celmatix Inc., New York, NY (US)

(72) Inventors: Piraye Yurttas BEIM, New York, NY (US); Anthony SANTISTEVAN, New York, NY (US)

(21) Appl. No.: 16/486,740

(22) PCT Filed: Feb. 8, 2018
(86) PCT No.: PCT/US2018/017402

§ 371 Date: Aug. 16, 2019

Related U.S. Application Data
(60) Provisional application No. 62/460,415, filed on Feb. 17, 2017.

(51) Int. Cl. G01N 33/76; A61K 38/09; G01N 33/68; C12M 3/00; C12Q 1/6883; A61K 38/24; G01G 19/50; A61P 15/08; G16H 20/10; G16H 10/40

(52) U.S. Cl. CPC G01N 33/76; A61K 38/09; G01N 33/689; C12M 21/06; G16H 10/40; A61K 38/24; G01G 19/50; A61P 15/08; G16H 20/10; C12Q 1/6883

(57) ABSTRACT

The invention provides methods for analyzing a patient's potential for achieving ongoing pregnancy with respect to a specific fertility treatment. These methods involve conducting one or more tests on the patient to determine a body mass index (BMI) of the patient and obtaining one or more clinical characteristics from the patient. In one aspect, at least one of the clinical characteristics is age, clinical diagnosis, weight, basal antral follicle count, or current medications. The methods further involve comparing the obtained clinical characteristics to a reference set of data obtained from a female reference population for which results of fertility treatments are known, at least some of the reference population having used ovulation induction agents. Based on the results of the comparing step, the patient is then informed of her potential for achieving ongoing pregnancy with respect to the specific fertility treatment.

### CROSS-REFERENCE TO RELATED APPLICATIONS

This application claims the benefit of and priority to U.S. Provisional Application Ser. No. 62/460,415, filed Feb. 17, 2017, the entire contents of which are incorporated herein by reference.

### BACKGROUND

Approximately one in seven couples has difficulty conceiving. Infertility may be due to a single cause or a combination of factors (e.g., genetic factors, diseases, or environmental factors) that prevents a pregnancy from occurring or continuing.

When a couple seeks medical assistance for reproductive difficulties, the couple is advised to undergo a number of diagnostic procedures to ascertain potential causes. Often the procedures can be highly invasive, costly, and time consuming. Moreover, traditional diagnostic procedures may not be informative of the ultimate chance of achieving live birth. The uncertainty surrounding prognosis and treatment decisions is a significant challenge for fertility specialists.

A number of other factors add to the complexity around diagnosing and treating infertility. For example, obesity has been associated in some studies with reduced success in achieving pregnancy. Understanding the differential impact of obesity on the success or failure of different types of fertility treatments, in different individuals would be helpful in guiding couples and women experiencing fertility difficulties to better outcomes.

### SUMMARY

The invention provides methods and systems for assessing a patient's ability of achieving ongoing pregnancy and for informing course of treatment. The invention provides methods for generating a likelihood of achieving pregnancy and for selecting appropriate treatment to promote successful pregnancy by accounting for the body mass index (BMI) of the patient as an element of increasing the likelihood of a successful pregnancy. The invention recognizes that

BMI plays an important role in a patient's ability to achieve ongoing pregnancy and impacts the selection and success of fertility treatment protocols, as determined through several large-scale studies detailed below in the description of the invention.

Methods of the invention involve the prospective likelihood of success of a selected fertility/infertility treatment based upon a number of factors indicative of the potential for achieving ongoing pregnancy with respect to a specific fertility treatment and then informing the patient of a recommended fertility treatment. Factors to be considered in the clinical algorithm include BMI, age, weight, basal antral follicle count (BAFC), current medications, basal follicle stimulating hormone (FSH) levels, progesterone levels, estrogen levels, AMH levels, total gonadotropin used, infertility diagnosis, parity, number of oocytes retrieved, embryo stage at transfer, number of usable embryos, number of embryos transferred, and clinic. In one aspect, methods involve the steps of conducting one or more tests on the patient to determine a body mass index, obtaining one or more clinical characteristics from the patient, comparing the results obtained from the obtaining and conducting steps to a reference set of data obtained from a female reference population for which results of fertility treatments are known, and informing the patient of the potential for achieving ongoing pregnancy with respect to a specific fertility treatment, wherein the potential is determined based on the comparing step. At least one of the clinical characteristics includes the patient's age, clinical diagnoses, weight, BAFC, current medications, basal FSH, estrogen levels, progesterone levels, AMH, total gonadotropin used, infertility diagnosis, parity, infertility diagnosis, total gonadotropin used, number of oocytes retrieved, embryo stage at transfer, number of usable embryos, number of embryos transferred, and/or clinic.

In one aspect, the fertility treatment is intrauterine insemination (IUI). The fertility treatment can also include the use of one or more ovulation induction agents. These ovulation induction agents include gonadotropins (sometimes referred to as “mini-stim”), such as FSH, luteinizing hormone (LH), and human chorionic gonadotropin (hCG); and oral ovulation induction agents. Exemplary oral ovulation induction agents include, but are not limited to: clomiphene citrate; aromatase inhibitors, such as letrozole and anastrozole; insulin sensitizing drugs, such as metformin, rosiglitazone, and pioglitazone; bromocriptine; cabergoline; gonadotropin-releasing hormone (GnRH); and GnRH analogs, such as leuprolide acetate, nafarelin acetate, goserelin acetate, ganirelix, and cetrorelix acetate (the former three being agonists and the latter two being antagonists); and combinations thereof. Whether gonadotropins alone or gonadotropins with oral ovulation induction agents are recommended, is dependent, in part, upon the patient's BMI. In another embodiment, the fertility treatment is an assisted reproductive technology (ART), such as in vitro fertilization (IVF) or intracytoplasmic sperm injection (ICSI).

In addition to clinical data, genetic data can also be included in generating a probability of achieving ongoing pregnancy. Genetic data, such as mutations in fertility-related genes and gene expression profiles, can be obtained from the patient and used in the generation of the probability for achieving ongoing pregnancy. In one aspect, the genetic data is compared to data from the reference population, which includes both clinical and genetic data, in order to provide the probability of achieving ongoing success.

In another embodiment, a method for treating a patient suspected of having impaired fertility is provided. The method involves the steps of conducting one or more tests on the patient to determine BMI of the patient; administering a gonadotropin along if the patient's BMI is near or above a threshold BMI, or a gonadotropin and optionally an oral ovulation induction agent if the patient's BMI is below the threshold BMI; and subjecting the patient to a fertility treatment. In one aspect, the fertility treatment is IUI. In another aspect, the gonadotropin includes one or more of LH, FSH, and/or hCG. In yet another aspect, the oral ovulation induction agent can be letrozole, clomiphene citrate, bromocriptine, metformin, or cabergoline. The threshold limit can be 20, 25, or 30.

### BRIEF DESCRIPTION OF DRAWINGS

FIG. 1 depicts female reproduction/fertility related functional biological classifications.

FIG. 2 depicts male reproduction/fertility related functional biological classifications.

FIG. 3 depicts spermatogenic functional biological classifications.

FIG. 4 represents a diagram of a system of the invention.

FIG. 5 depicts the effects of BMI on the predicted probability of achieving ongoing pregnancy.

FIG. 6 depicts the effects of BMI on the predicted probability of achieving ongoing pregnancy with gonadotropin and oral agents, and without.

FIG. 7A depicts the effects of BMI on cycle cancellation.

FIG. 7B depicts the effects of BMI on the number of oocytes retrieved.

FIG. 8A depicts the effects of BMI on the number of usable embryos.

FIG. 8B depicts the effects of BMI on ongoing clinical pregnancy.

### DETAILED DESCRIPTION

The invention relates to methods and systems for assessing likelihood (i.e., probability) of achieving pregnancy and for therapeutic intervention to achieve pregnancy. The invention provides methods for generating a likelihood of achieving pregnancy and guiding treatment therefrom using clinical data and specifically accounting for the body mass index (BMI) of the patient. In accordance with the present invention, the generation of a likelihood of achieving ongoing pregnancy for a certain fertility treatment in an individual takes into consideration clinical data, and optionally genetic data. The methods involve the determination of one or more correlations between clinical characteristics and known pregnancy and infertility-related outcomes from a reference set of data to provide a model representative of a cumulative probability of ongoing pregnancy for a certain fertility treatment. The methods can further involve the determination of one or more correlations between genetic characteristics and known pregnancy and infertility-related outcomes from the reference set of data to adjust the model. The model can then be applied to the patient's data to generate the likelihood of achieving ongoing pregnancy in the subject for a certain fertility treatment. Based on the generated likelihood, a treatment protocol can be recommended. As discussed in more detail below, the treatment protocol may depend, in part, on the BMI of the patient.

### Clinical Data

Assessment and analysis of the potential for achieving ongoing pregnancy and live birth incorporates the use of clinical fertility-associated information, or data, such as phenotypic and/or environmental characteristics. Exemplary clinical information is provided in Table 1 below.

TABLE 1
Clinical information

Cholesterol levels on different days of the menstrual cycle
Age of first menses for patient and female blood relatives (e.g. sisters, mother, grandmothers)
Age of menopause for female blood relatives (e.g. sisters, mother, grandmothers)
Number of previous pregnancies (gravidity and parity; biochemical/ectopic/clinical/fetal heart
beat detected, live birth outcomes), age at the time, and outcome for patient and female blood
relatives (e.g. sisters, mother, grandmothers
Diagnosis of polycystic ovary syndrome
Basal antral follicular count (BAFC)
number of embryos transferred
PGS
Female hormone levels, such as, anti-Mullerian hormone (AMH), luteinizing hormone (LH),
follicle stimulating hormone (FSH), Progesterone and Estrogens (including E₁, estrone; E₂,
estradiol; E₃ estriol)
History of hydrosalpinx or tubal occlusion
History of endometriosis, pelvic pain, or painful periods
Cancer history/type of cancer/treatment/outcome for patient and female blood relatives (e.g.
sisters, mother, grandmothers)
Age that sexual activity began, current level of sexual activity
Smoking history for patient and blood relatives
Travel schedule/number of flying hours a year/time difference changes of more than 3 hours
(jetlag and flight-associated radiation exposure)
Nature of periods (length of menses, length of cycle)
Biological age (number of years since first menses)
Birth control use
Drug use (illegal or legal)
Body mass index (BMI; current, lowest ever, highest ever)
History of polyps
History of hormonal imbalance
History of amenorrhoea
History of eating disorders
Alcohol consumption by patient or blood relatives
Details of mother's pregnancy with patient (i.e. measures of uterine environment): any drugs
taken, smoking, alcohol, stress levels, exposure to plastics (i.e. Tupperware), composition of
diet (see below)
Sleep patterns: number of hours a night, continuous/overall
Diet: meat, organic produce, vegetables, vitamin or other supplement consumption, dairy (full
fat or reduced fat), coffee/tea consumption, folic acid, sugar (complex, artificial, simple),
processed food versus home cooked
Exposure to plastics: microwave in plastic, cook with plastic, store food in plastic, plastic water
or coffee mugs
Water consumption: amount per day, format: straight from the tap, bottled water (plastic or
bottle), filtered (type: e.g. Britta/Pur)
Residence history starting with mother's pregnancy: location/duration
Environmental exposure to potential toxins for different regions (extracted from government
monitoring databases)
Health metrics: autoimmune disease, chronic illness/condition
Pelvic surgery history
Life time number of pelvic X-rays
History of sexually transmitted infections: type/treatment/outcome
Stress
Thickness and type of endometrium throughout the menstrual cycle
Age
Height
Fertility treatment history and details: history of hormone stimulation, brand of drugs used,
BAFC, follicle count after stimulation with different protocols, number/quality/stage of
retrieved oocytes/development profile of embryos resulting from in vitro insemination (natural
or ICSI), details of IVF procedure (which clinic, doctor/embryologist at clinic, assisted
hatching, fresh or thawed oocytes/embryos, embryo transfer (blood on the catheter/squirt
detection and direction on ultrasound), number of successful and unsuccessful IVF attempts
Morning sickness during pregnancy
Breast size before/during/after pregnancy
History of ovarian cysts
Twin or sibling from multiple birth (mono-zygotic or di-zygotic)
Semen analysis (count, motility, morphology)
Vasectomy
Testosterone levels
Date of last use and/or frequency of use of a hot tub or sauna
Blood type
DES exposure in utero
Past and current exercise/athletic history
Levels of phthalates, including metabolites:
MEP—monoethyl phthalate, MECPP—mono(2-ethyl-5-carboxypentyl) phthalate,
MEHHP—mono(2-ethyl-5-hydroxyhexyl) phthalate, MEOHP—mono(2-ethyl-5-ox-ohexyl) phthalate,
MBP—monobutyl phthalate, MBzP—monobenzyl phthalate, MEHP—mono(2-ethylhexyl)
phthalate, MiBP—mono-isobutyl phthalate, MCPP—mono(3-carboxypropyl) phthalate,
MCOP—monocarboxyisooctyl phthalate, MCNP—monocarboxyisononyl phthalate
Familial history of primary ovarian insufficiency
Autoimmunity history - Antiadrenal antibodies (anti-21-hydroxylase antibodies), antiovarian
antibodies, antithyroid antibodies (anti-thyroid peroxidase, antithyroglobulin)
Additional female hormone levels Δ4-Androstenedione (using radioimmunoassay),
Dehydroepiandrosterone (using radioimmunoassay), and Inhibin B (commercial ELISA)
Number of years trying to conceive
Dioxin and PVC exposure
Hair color
Nevi (moles)
Lead, cadmium, and other heavy metal exposure
For a particular ART cycle: the percentage of eggs that were abnormally fertilized, if assisted
hatching was performed, if anesthesia was used, average number of cells contained by the
embryo at the time of cryopreservation, average degree of expansion for blastocyst represented
as a score, average degree of expansion of a previously frozen embryo represented as a score,
embryo quality metrics including but not limited to degree of cell fragmentation and
visualization of a or organization/number of cells contained in the inner cell mass (ICM), the
fraction of overall embryos that make it to the blastocyst stage of development, the number of
embryos that make it to the blastocyst stage of development, use of birth control, the brand
name of the hormones used in ovulation induction, hyperstimulation syndrome, reason for
cancelation of a treatment cycle, chemical pregnancy detected, clinical pregnancy detected,
count of germinal vesicle containing oocytes upon retrieval, count of metaphase I stage eggs
upon retrieval, count of metaphase II stage eggs upon retrieval, count of embryos or oocytes
arrested in development and the stage of development or day of development post oocyte
retrieval, number of embryos transferred and date in days post-oocyte retrieval that the embryos
were transferred, how many embryos were cryopreserved and at what stage of development

In one embodiment, the assessment of a patient's probability of achieving an ongoing pregnancy incorporates clinical data such as age, BAFC, medication type, sperm motility, clinical diagnoses, BMI, hormone levels, and previous fertility treatments (including the use of ovulation induction agents).

Clinical information can be obtained by any means known in the art. In many cases this information can be obtained from a questionnaire completed by the subject that contains questions regarding certain clinical data, such as age. Additional information can be obtained from a questionnaire completed by the subject's partner and blood relatives. The questionnaire includes questions regarding the subject's clinical traits, such as his or her age, smoking habits, or frequency of alcohol consumption.

Information can also be obtained from the medical history of the subject, as well as the medical history of blood relatives and other family members, such as any clinical diagnoses, prior fertility treatments and current medications. Additional information can be obtained from the medical history and family medical history of the subject's partner. Medical history information can be obtained through analysis of electronic medical records, paper medical records, a series of questions about medical history included in the questionnaire, and a combination thereof.

In other embodiments, an assay specific to a phenotypic trait or an environmental exposure of interest is used. Such assays are known to those of skill in the art, and may be used with methods of the invention. For example, hormones, such as FSH and LH, may be detected from a urine or blood test. Venners et al. (Hum. Reprod. 21(9): 2272-2280, 2006) reports assays for detecting estrogen and progesterone in urine and blood samples. Venners also reports assays for detecting the chemicals used in fertility treatments.

Illicit drug use may be detected from a tissue or body fluid, such as hair, urine, sweat, or blood, and there are numerous commercially available assays (LabCorp) for conducting such tests. Standard drug tests look for ten different classes of drugs, and the test is commercially known as a “10-panel urine screen”. The 10-panel urine screen consists of the following: 1. Amphetamines (including Methamphetamine) 2. Barbiturates 3. Benzodiazepines 4.

Cannabinoids (THC) 5. Cocaine 6. Methadone 7. Methaqualone 8. Opiates (Codeine, Morphine, Heroin, Oxycodone, Vicodin, etc.) 9. Phencyclidine (PCP) 10. Propoxyphene. Use of alcohol can also be detected by such tests.

Numerous assays can be used to test a patient's exposure to plastics (e.g., Bisphenol A (BPA)). BPA is most commonly found as a component of polycarbonates (about 74% of total

BPA produced) and in the production of epoxy resins (about 20%). As well as being found in a myriad of products including plastic food and beverage contains (including baby and water bottles), BPA is also commonly found in various household appliances, electronics, sports safety equipment, adhesives, cash register receipts, medical devices, eyeglass lenses, water supply pipes, and many other products. Assays for testing blood, sweat, or urine for presence of BPA are described, for example, in Genuis et al. (Journal of Environmental and Public Health, Volume 2012, Article ID 185731, 10 pages, 2012).

A subject's BMI can be determined by first obtaining the subject's weight and height and then comparing to or inputting that information into a physical or computer-based table or chart. BMI is a value derived from the mass and height of an individual that is used to quantify the amount of tissue mass (including muscle, fat, and bone) in an individual, such that the individual can be categorized as underweight, normal weight, overweight, or obese. The commonly accepted ranges can be found in Table 2 below.

TABLE 2
Commonly Accepted Body Mass Index Ranges

| Range | kg/m² |
|---|---|
| Underweight | <18.5 |
| Normal weight | 18.5-25 |
| Overweight | 25-30 |
| Obese | ≥30 |
| Obese class I | 30.00-34.99 |
| Obese class II | 35.00-39.99 |
| Obese class III | ≥40.00 |

Antral follicle count (AFC) can be determined through the use of ultrasound, preferably a vaginal ultrasound. Antral follicles are small follicles within the ovaries that present during a later stage of folliculogenesis. BAFC are often used as a biomarker for ovarian reserve.

### Genetic Data

In one aspect of the invention, the assessment of the patient's probability of achieving ongoing pregnancy and subsequent determination of a treatment protocol includes the use of genetic data from both the patient and a reference population. This genetic data is utilized to provide more accurate prognoses that can inform downstream diagnostic tests and treatments that may benefit the subject.

Genetic data for use with methods of the invention include any biomarkers that are associated with reproduction, infertility/ability to achieving ongoing pregnancy. Exemplary biomarkers include genes (e.g. any region of DNA encoding a functional product), genetic regions (e.g. regions including genes and intergenic regions with a particular focus on regions conserved throughout evolution in placental mammals), and gene products (e.g., RNA and protein). In certain embodiments, the biomarker is an infertility-associated gene or genetic region. A reproductive health-associated genetic region is any DNA sequence in which variation is associated with a change in reproductive health. Examples of changes in reproductive health include, but are not limited to, the following: a homozygous mutation of an infertility-associated gene leads to a complete loss of fertility; a homozygous mutation of an infertility-associated gene is incompletely penetrant and leads to reduction in fertility that varies from individual to individual; a heterozygous mutation is completely recessive, having no effect on fertility; and the infertility-associated gene is X-linked, such that a potential defect in fertility depends on whether a non-functional allele of the gene is located on an inactive X chromosome (Barr body) or on an expressed X chromosome.

In particular embodiments, the assessed infertility-associated genetic region is a maternal effect gene. Maternal effects genes are genes that have been found to encode key structures and functions in mammalian oocytes (Yurttas et al., Reproduction 139:809-823, 2010). Maternal effect genes are described, for example in, Christians et al. (Mol Cell Biol 17:778-88, 1997); Christians et al., Nature 407:693-694, 2000); Xiao et al. (EMBO J 18:5943-5952, 1999); Tong et al. (Endocrinology 145:1427-1434, 2004); Tong et al. (Nat Genet 26:267-268, 2000); Tong et al. (Endocrinology, 140:3720-3726, 1999); Tong et al. (Hum Reprod 17:903-911, 2002); Ohsugi et al. (Development 135:259-269, 2008); Borowczyk et al. (Proc Natl Acad Sci U S A., 2009); and

Wu (Hum Reprod 24:415-424, 2009). Maternal effects genes are also described in U.S. 12/889,304. The content of each of these is incorporated by reference herein in its entirety.

In particular embodiments, the reproductive health-associated genetic region is one or more genes (including exons, introns, and 10 kb of DNA flanking either side of said gene) selected from the genes shown in Table 3 below. In Table 3, OMIM reference numbers are provided when available.

TABLE 3
Human Infertility-Related Genes (OMIM #)

| | | | |
|---|---|---|---|
| ABCA1 (600046) | ACTL6A (604958) | ACTL8 | ACVR1 (102576) |
| ACVR1B (601300) | ACVR1C (608981) | ACVR2(102581) | ACVR2A (102581) |
| ACVR2B (602730) | ACVRL1 (601284) | ADA (608958) | ADAMTS1 (605174) |
| ADM (103275) | ADM2 (608682) | AFF2 (300806) | AGT (106150) |
| AHR (600253) | AIRE (607358) | AK2 (103020) | AK7 |
| AKR1C1 (600449) | AKR1C2 (600450) | AKR1C3 (603966) | AKR1C4 (600451) |
| AKT1 (164730) | ALDOA (103850) | ALDOB (612724) | ALDOC (103870) |
| ALPL (171760) | AMBP (176870) | AMD1 (180980) | AMH (600957) |
| AMHR2 (600956) | ANK3 (600465) | ANXA1 (151690) | APC (611731) |
| APOA1 (107680) | APOE (107741) | AQP4 (600308) | AR (313700) |
| AREG (104640) | ARF1 (103180) | ARF3 (103190) | ARF4 (601177) |
| ARF5 (103188) | ARFRP1 (604699) | ARL1 (603425) | ARL10 (612405) |
| ARL11 (609351) | ARL13A | ARL13B (608922) | ARL15 |
| ARL2 (601175) | ARL3 (604695) | ARL4A (604786) | ARL4C (604787) |
| ARL4D (600732) | ARL5A (608960) | ARL5B (608909) | ARL5C |
| ARL6 (608845) | ARL8A | ARL8B | ARMC2 |
| ARNTL (602550) | ASCL2 (601886) | ATF7IP (613644) | ATG7 (608760) |
| ATM (607585) | ATR (601215) | ATXN2 (601517) | AURKA (603072) |
| AURKB (604970) | AUTS2 (607270) | BARD1 (601593) | BAX (600040) |
| BBS1 (209901) | BBS10 (610148) | BBS12 (610683) | BBS2 (606151) |
| BBS4 (600374) | BBS5 (603650) | BBS7 (607590) | BBS9 (607968) |
| BCL2 (151430) | BCL2L1 (600039) | BCL2L10 (606910) | BDNF (113505) |
| BECN1 (604378) | BHMT (602888) | BLVRB (600941) | BMP15 (300247) |
| BMP2 (112261) | BMP3 (112263) | BMP4 (112262) | BMP5 (112265) |
| BMP6 (112266) | BMP7 (112267) | BMPR1A (601299) | BMPR1B (603248) |
| BMPR2 (600799) | BNC1 (601930) | BOP1 (610596) | BRCA1 (113705) |
| BRCA2 (600185) | BRIP1 (605882) | BRSK1 (609235) | BRWD1 |
| BSG (109480) | BTG4 (605673) | BUB1 (602452) | BUB1B (602860) |
| C2orf86 (613580) | C3 (120700) | C3orf56 | C6orf221 (611687) |
| CA1 (114800) | CARD8 (609051) | CARM1 (603934) | CASP1 (147678) |
| CASP2 (600639) | CASP5 (602665) | CASP6 (601532) | CASP8 (601763) |
| CBS (613381) | CBX1 (604511) | CBX2 (602770) | CBX5 (604478) |
| CCDC101 (613374) | CCDC28B (610162) | CCL13 (601391) | CCL14 (601392) |
| CCL4 (182284) | CCL5 (187011) | CCL8 (602283) | CCND1 (168461) |
| CCND2 (123833) | CCND3 (123834) | CCNH (601953) | CCS (603864) |
| CD19 (107265) | CD24 (600074) | CD55 (125240) | CD81 (186845) |
| CD9 (143030) | CDC42 (116952) | CDK4 (123829) | CDK6 (603368) |
| CDK7 (601955) | CDKN1B (600778) | CDKN1C (600856) | CDKN2A (600160) |
| CDX2 (600297) | CDX4 (300025) | CEACAM20 | CEBPA (116897) |
| CEBPB (189965) | CEBPD (116898) | CEBPE (600749) | CEBPG (138972) |
| CEBPZ (612828) | CELF1 (601074) | CELF4 (612679) | CENPB (117140) |
| CENPF (600236) | CENPI (300065) | CEP290 (610142) | CFC1 (605194) |
| CGA (118850) | CGB (118860) | CGB1 (608823) | CGB2 (608824) |
| CGB5 (608825) | CHD7 (608892) | CHST2 (603798) | CLDN3 (602910) |
| COIL (600272) | COL1A2 (120160) | COL4A3BP | COMT (116790) |
| | | (604677) | |
| COPE (606942) | COX2 (600262) | CP (117700) | CPEB1 (607342) |
| CRHR1 (122561) | CRYBB2 (123620) | CSF1 (120420) | CSF2 (138960) |
| CSTF1 (600369) | CSTF2 (600368) | CTCF (604167) | CTCFL (607022) |
| CTF2P | CTGF (121009) | CTH (607657) | CTNNB1 (116806) |
| CUL1 (603134) | CX3CL1 (601880) | CXCL10 (147310) | CXCL9 (601704) |
| CXorf67 | CYP11A1 (118485) | CYP11B1 (610613) | CYP11B2 (124080) |
| CYP17A1 (609300) | CYP19A1 (107910) | CYP1A1 (108330) | CYP27B1 (609506) |
| DAZ2 (400026) | DAZL (601486) | DCTPP1 | DDIT3 (126337) |
| DDX11 (601150) | DDX20 (606168) | DDX3X (300160) | DDX43 (606286) |
| DEPDC7 (612294) | DHFR (126060) | DHFRL1 | DIAPH2 (300108) |
| DICER1 (606241) | DKK1 (605189) | DLC1 (604258) | DLGAP5 |
| DMAP1 (605077) | DMC1 (602721) | DNAJB1 (604572) | DNMT1 (126375) |
| DNMT3B (602900) | DPPA3 (608408) | DPPA5 (611111) | DPYD (612779) |
| DTNBP1 (607145) | DYNLL1 (601562) | ECHS1 (602292) | EEF1A1 (130590) |
| EEF1A2 (602959) | EFNA1 (191164) | EFNA2 (602756) | EFNA3 (601381) |
| EFNA4 (601380) | EFNA5 (601535) | EFNB1 (300035) | EFNB2 (600527) |
| EFNB3 (602297) | EGR1 (128990) | EGR2 (129010) | EGR3 (602419) |
| EGR4 (128992) | EHMT1 (607001) | EHMT2 (604599) | EIF2B2 (606454) |
| EIF2B4 (606687) | EIF2B5 (603945) | EIF2C2 (606229) | EIF3C (603916) |
| EIF3CL (603916) | EPHA1 (179610) | EPHA10 (611123) | EPHA2 (176946) |
| EPHA3 (179611) | EPHA4 (602188) | EPHA5 (600004) | EPHA6 (600066) |
| EPHA7 (602190) | EPHA8 (176945) | EPHB1 (600600) | EPHB2 (600997) |
| EPHB3 (601839) | EPHB4 (600011) | EPHB6 (602757) | ERCC1 (126380) |
| ERCC2 (126340) | EREG (602061) | ESR1 (133430) | ESR2 (601663) |
| ESR2 (601663) | ESRRB (602167) | ETV5 (601600) | EZH2 (601573) |
| EZR (123900) | FANCC (613899) | FANCG (602956) | FANCL (608111) |
| FAR1 | FAR2 | FASLG (134638) | FBN1 (134797) |
| FBN2 (612570) | FBN3 (608529) | FBRS (608601) | FBRSL1 |
| FBXO10 (609092) | FBXO11 (607871) | FCRL3 (606510) | FDXR (103270) |
| FGF23 (605380) | FGF8 (600483) | FGFBP1 (607737) | FGFBP3 |
| FGFR1 (136350) | FHL2 (602633) | FIGLA (608697) | FILIP1L (612993) |
| FKBP4 (600611) | FMN2 (606373) | FMR1 (309550) | FOLR1 (136430) |
| FOLR2 (136425) | FOXE1 (602617) | FOXL2 (605597) | FOXN1 (600838) |
| FOXO3 (602681) | FOXP3 (300292) | FRZB (605083) | FSHB (136530) |
| FSHR (136435) | FST (136470) | GALT (606999) | GBP5 (611467) |
| GCK (138079) | GDF1 (602880) | GDF3 (606522) | GDF9 (601918) |
| GGT1 (612346) | GJA1 (121014) | GJA10 (611924) | GJA3 (121015) |
| GJA4 (121012) | GJA5 (121013) | GJA8 (600897) | GJB1 (304040) |
| GJB2 (121011) | GJB3 (603324) | GJB4 (605425) | GJB6 (604418) |
| GJB7 (611921) | GJC1 (608655) | GJC2 (608803) | GJC3 (611925) |
| GJD2 (607058) | GJD3 (607425) | GJD4 (611922) | GNA13 (604406) |
| GNB2 (139390) | GNRH1 (152760) | GNRH2 (602352) | GNRHR (138850) |
| GPC3 (300037) | GPRC5A (604138) | GPRC5B (605948) | GREM2 (608832) |
| GRN (138945) | GSPT1 (139259) | GSTA1 (138359) | H19 (103280) |
| H1FOO (142709) | HABP2 (603924) | HADHA (600890) | HAND2 (602407) |
| HBA1 (141800) | HBA2 (141850) | HBB (141900) | HELLS (603946) |
| HK3 (142570) | HMOX1 (141250) | HNRNPK (600712) | HOXA11 (142958) |
| HPGD (601688) | HS6ST1 (604846) | HSD17B1 (109684) | HSD17B12 (609574) |
| HSD17B2 (109685) | HSD17B4 (601860) | HSD17B7 (606756) | HSD3B1 (109715) |
| HSF1 (140580) | HSF2BP (604554) | HSP90B1 (191175) | HSPG2 (142461) |
| HTATIP2 (605628) | ICAM1 (147840) | ICAM2 (146630) | ICAM3 (146631) |
| IDH1 (147700) | IFI30 (604664) | IFITM1 (604456) | IGF1 (147440) |
| IGF1R (147370) | IGF2 (147470) | IGF2BP1 (608288) | IGF2BP2 (608289) |
| IGF2BP3 (608259) | IGF2BP3 (608259) | IGF2R (147280) | IGFALS (601489) |
| IGFBP1 (146730) | IGFBP2 (146731) | IGFBP3 (146732) | IGFBP4 (146733) |
| IGFBP5 (146734) | IGFBP6 (146735) | IGFBP7 (602867) | IGFBPL1 (610413) |
| IL10 (124092) | IL11RA (600939) | IL12A (161560) | IL12B (161561) |
| IL13 (147683) | IL17A (603149) | IL17B (604627) | IL17C (604628) |
| IL17D (607587) | IL17F (606496) | IL1A (147760) | IL1B (147720) |
| IL23A (605580) | IL23R (607562) | IL4 (147780) | IL5 (147850) |
| IL5RA (147851) | IL6 (147620) | IL6ST (600694) | IL8 (146930) |
| ILK (602366) | INHA (147380) | INHBA (147290) | INHBB (147390) |
| IRF1 (147575) | ISG15 (147571) | ITGA11 (604789) | ITGA2 (192974) |
| ITGA3 (605025) | ITGA4 (192975) | ITGA7 (600536) | ITGA9 (603963) |
| ITGAV (193210) | ITGB1 (135630) | JAG1 (601920) | JAG2 (602570) |
| JARID2 (601594) | JMY (604279) | KAL1 (300836) | KDM1A (609132) |
| KDM1B (613081) | KDM3A (611512) | KDM4A (609764) | KDM5A (180202) |
| KDM5B (605393) | KHDC1 (611688) | KIAA0430 (614593) | KIF2C (604538) |
| KISS1 (603286) | KISS1R (604161) | KITLG (184745) | KL (604824) |
| KLF4 (602253) | KLF9 (602902) | KLHL7 (611119) | LAMC1 (150290) |
| LAMC2 (150292) | LAMP1 (153330) | LAMP2 (309060) | LAMP3 (605883) |
| LDB3 (605906) | LEP (164160) | LEPR (601007) | LFNG (602576) |
| LHB (152780) | LHCGR (152790) | LHX8 (604425) | LIF (159540) |
| LIFR (151443) | LIMS1 (602567) | LIMS2 (607908) | LIMS3 |
| LIMS3L | LIN28 (611043) | LIN28B (611044) | LMNA (150330) |
| LOC613037 | LOXL4 (607318) | LPP (600700) | LYRM1 (614709) |
| MAD1L1 (602686) | MAD2L1 (601467) | MAD2L1BP | MAF (177075) |
| MAP3K1 (600982) | MAP3K2 (609487) | MAPK1 (176948) | MAPK3 (601795) |
| MAPK8 (601158) | MAPK9 (602896) | MB21D1 (613973) | MBD1 (156535) |
| MBD2 (603547) | MBD3 (603573) | MBD4 (603574) | MCL1 (159552) |
| MCM8 (608187) | MDK (162096) | MDM2 (164785) | MDM4 (602704) |
| MECP2 (300005) | MED12 (300188) | MERTK (604705) | METTL3 (612472) |
| MGAT1 (160995) | MITF (156845) | MKKS (604896) | MKS1 (609883) |
| MLH1 (120436) | MLH3 (604395) | MOS (190060) | MPPED2 (600911) |
| MRS2 | MSH2 (609309) | MSH3 (600887) | MSH4 (602105) |
| MSH5 (603382) | MSH6 (600678) | MST1 (142408) | MSX1 (142983) |
| MSX2 (123101) | MTA2 (603947) | MTHFD1 (172460) | MTHFR (607093) |
| MTO1 (614667) | MTOR (601231) | MTRR (602568) | MUC4 (158372) |
| MVP (605088) | MX1 (147150) | MYC (190080) | NAB1 (600800) |
| NAB2 (602381) | NAT1 (108345) | NCAM1 (116930) | NCOA2 (601993) |
| NCOR1 (600849) | NCOR2 (600848) | NDP (300658) | NFE2L3 (604135) |
| NLRP1 (606636) | NLRP10 (609662) | NLRP11 (609664) | NLRP12 (609648) |
| NLRP13 (609660) | NLRP14 (609665) | NLRP2 (609364) | NLRP3 (606416) |
| NLRP4 (609645) | NLRP5 (609658) | NLRP6 (609650) | NLRP7 (609661) |
| NLRP8 (609659) | NLRP9 (609663) | NNMT (600008) | NOBOX (610934) |
| NODAL (601265) | NOG (602991) | NOS3 (163729) | NOTCH1 (190198) |
| NOTCH2 (600275) | NPM2 (608073) | NPR2 (108961) | NR2C2 (601426) |
| NR3C1 (138040) | NR5A1 (184757) | NR5A2 (604453) | NRIP1 (602490) |
| NRIP2 | NRIP3 (613125) | NTF4 (162662) | NTRK1 (191315) |
| NTRK2 (600456) | NUPR1 (614812) | OAS1 (164350) | OAT (613349) |
| OFD1 (300170) | OOEP (611689) | ORAI1 (610277) | OTC (300461) |
| PADI1 (607934) | PADI2 (607935) | PADI3 (606755) | PADI4 (605347) |
| PADI6 (610363) | PAEP (173310) | PAIP1 (605184) | PARP12 (612481) |
| PCNA (176740) | PCP4L1 | PDE3A (123805) | PDK1 (602524) |
| PGK1 (311800) | PGR (607311) | PGRMC1 (300435) | PGRMC2 (607735) |
| PIGA (311770) | PIM1 (164960) | PLA2G2A (172411) | PLA2G4C (603602) |
| PLA2G7 (601690) | PLAC1L | PLAG1 (603026) | PLAGL1 (603044) |
| PLCB1 (607120) | PMS1 (600258) | PMS2 (600259) | POF1B (300603) |
| POLG (174763) | POLR3A (614258) | POMZP3 (600587) | POU5F1 (164177) |
| PPID (601753) | PPP2CB (176916) | PRDM1 (603423) | PRDM9 (609760) |
| PRKCA (176960) | PRKCB (176970) | PRKCD (176977) | PRKCDBP |
| PRKCE (176975) | PRKCG (176980) | PRKCQ (600448) | PRKRA (603424) |
| PRLR (176761) | PRMT1 (602950) | PRMT10 (307150) | PRMT2 (601961) |
| PRMT3 (603190) | PRMT5 (604045) | PRMT6 (608274) | PRMT7 (610087) |
| PRMT8 (610086) | PROK1 (606233) | PROK2 (607002) | PROKR1 (607122) |
| PROKR2 (607123) | PSEN1 (104311) | PSEN2 (600759) | PTGDR (604687) |
| PTGER1 (176802) | PTGER2 (176804) | PTGER3 (176806) | PTGER4 (601586) |
| PTGES (605172) | PTGES2 (608152) | PTGES3 (607061) | PTGFR (600563) |
| PTGFRN (601204) | PTGS1 (176805) | PTGS2 (600262) | PTN (162095) |
| PTX3 (602492) | QDPR (612676) | RAD17 (603139) | RAX (601881) |
| RBP4 (180250) | RCOR1 (607675) | RCOR2 | RCOR3 |
| RDH11 (607849) | REC8 (608193) | REXO1 (609614) | REXO2 (607149) |
| RFPL4A (612601) | RGS2 (600861) | RGS3 (602189) | RSPO1 (609595) |
| RTEL1 (608833) | SAFB (602895) | SAR1A (607691) | SAR1B (607690) |
| SCARB1 (601040) | SDC3 (186357) | SELL (153240) | SEPHS1 (600902) |
| SEPHS2 (606218) | SERPINA10 | SFRP1 (604156) | SFRP2 (604157) |
| | (605271) | | |
| SFRP4 (606570) | SFRP5 (604158) | SGK1 (602958) | SGOL2 (612425) |
| SH2B1 (608937) | SH2B2 (605300) | SH2B3 (605093) | SIRT1 (604479) |
| SIRT2 (604480) | SIRT3 (604481) | SIRT4 (604482) | SIRT5 (604483) |
| SIRT6 (606211) | SIRT7 (606212) | SLC19A1 (600424) | SLC28A1 (606207) |
| SLC28A2 (606208) | SLC28A3 (608269) | SLC2A8 (605245) | SLC6A2 (163970) |
| SLC6A4 (182138) | SLCO2A1 (601460) | SLITRK4 (300562) | SMAD1 (601595) |
| SMAD2 (601366) | SMAD3 (603109) | SMAD4 (600993) | SMAD5 (603110) |
| SMAD6 (602931) | SMAD7 (602932) | SMAD9 (603295) | SMARCA4 (603254) |
| SMARCA5 (603375) | SMC1A (300040) | SMC1B (608685) | SMC3 (606062) |
| SMC4 (605575) | SMPD1 (607608) | SOCS1 (603597) | SOD1 (147450) |
| SOD2 (147460) | SOD3 (185490) | SOX17 (610928) | SOX3 (313430) |
| SPAG17 | SPARC (182120) | SPIN1 (609936) | SPN (182160) |
| SPO11 (605114) | SPP1 (166490) | SPSB2 (611658) | SPTB (182870) |
| SPTBN1 (182790) | SPTBN4 (606214) | SRCAP (611421) | SRD5A1 (184753) |
| SRSF4 (601940) | SRSF7 (600572) | ST5 (140750) | STAG3 (608489) |
| STAR (600617) | STARD10 | STARD13 (609866) | STARD3 (607048) |
| STARD3NL | STARD4 (607049) | STARD5 (607050) | STARD6 (607051) |
| (611759) | | | |
| STARD7 | STARD8 (300689) | STARD9 (614642) | STAT1 (600555) |
| STAT2 (600556) | STAT3 (102582) | STAT4 (600558) | STAT5A (601511) |
| STAT5B (604260) | STAT6 (601512) | STC1 (601185) | STIM1 (605921) |
| STK3 (605030) | SULT1E1 (600043) | SUZ12 (606245) | SYCE1 (611486) |
| SYCE2 (611487) | SYCP1 (602162) | SYCP2 (604105) | SYCP3 (604759) |
| SYNE1 (608441) | SYNE2 (608442) | TAC3 (162330) | TACC3 (605303) |
| TACR3 (162332) | TAF10 (600475) | TAF3 (606576) | TAF4 (601796) |
| TAF4B (601689) | TAF5 (601787) | TAF5L | TAF8 (609514) |
| TAF9 (600822) | TAP1 (170260) | TBL1X (300196) | TBXA2R (188070) |
| TCL1A (186960) | TCL1B (603769) | TCL6 (604412) | TCN2 (613441) |
| TDGF1 (187395) | TERC (602322) | TERF1 (600951) | TERT (187270) |
| TEX12 (605791) | TEX9 | TF (190000) | TFAP2C (601602) |
| TFPI (152310) | TFPI2 (600033) | TG (188450) | TGFB1 (190180) |
| TGFB1I1 (602353) | TGFBR3 (600742) | THOC5 (612733) | THSD7B |
| TLE6 (612399) | TM4SF1 (191155) | TMEM67 (609884) | TNF (191160) |
| TNFAIP6 (600410) | TNFSF13B (603969) | TOP2A (126430) | TOP2B (126431) |
| TP53 (191170) | TP53I3 (605171) | TP63 (603273) | TP73 (601990) |
| TPMT (187680) | TPRXL (611167) | TPT1 (600763) | TRIM32 (602290) |
| TSC2 (191092) | TSHB (188540) | TSIX (300181) | TTC8 (608132) |
| TUBB4Q (158900) | TUFM (602389) | TYMS (188350) | UBB (191339) |
| UBC (191340) | UBD (606050) | UBE2D3 (602963) | UBE3A (601623) |
| UBL4A (312070) | UBL4B (611127) | UIMC1 (609433) | UQCR11 (609711) |
| UQCRC2 (191329) | USP9X (300072) | VDR (601769) | VEGFA (192240) |
| VEGFB (601398) | VEGFC (601528) | VHL (608537) | VIM (193060) |
| VKORC1 (608547) | VKORC1L1 | WAS (300392) | WISP2 (603399) |
| | (608838) | | |
| WNT7A (601570) | WNT7B (601967) | WT1 (607102) | XDH (607633) |
| XIST (314670) | YBX1 (154030) | YBX2 (611447) | ZAR1 (607520) |
| ZFX (314980) | ZNF22 (194529) | ZNF267 (604752) | ZNF689 |
| ZNF720 | ZNF787 | ZNF84 | ZP1 (195000) |
| ZP2 (182888) | ZP3 (182889) | ZP4 (613514) | |

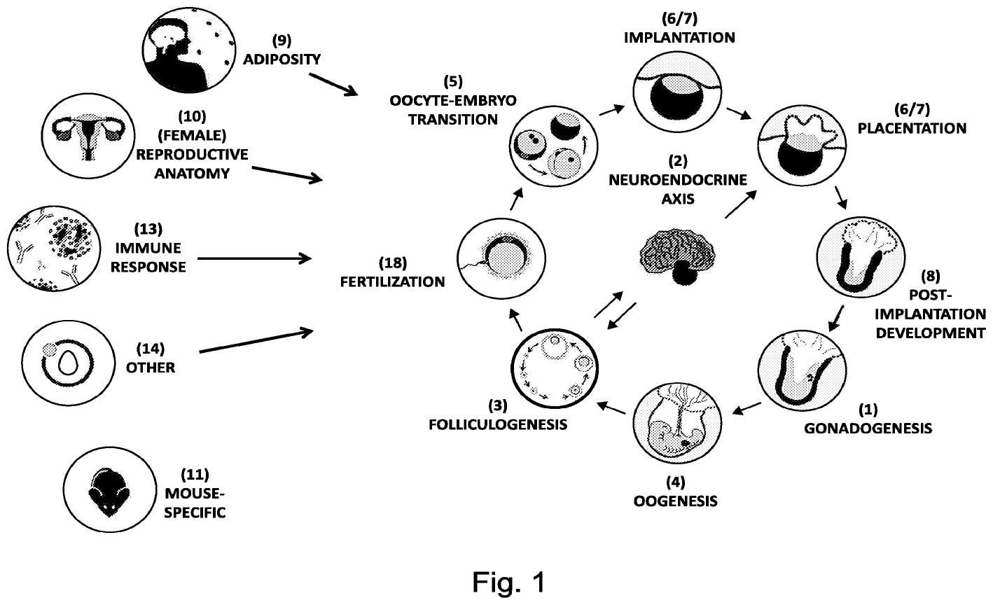

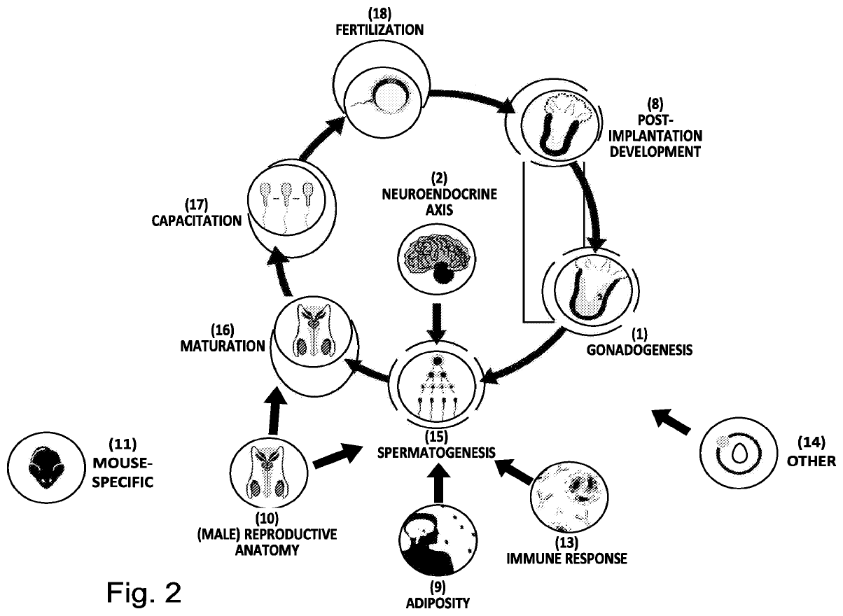

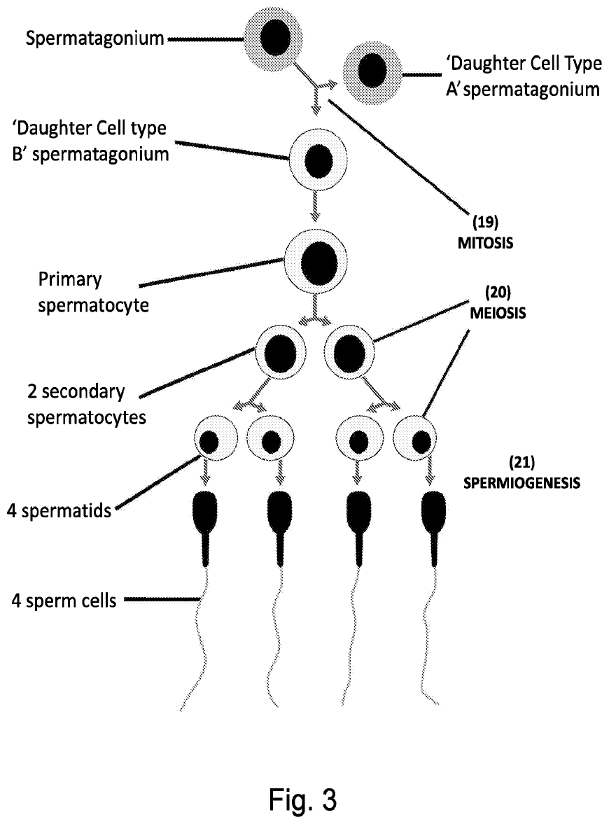

The genes listed in Table 3 can be involved in different aspects of reproduction/fertility related processes. Furthermore, additional genes beyond those maternal effect genes listed in Table 3 can also affect fertility. Genes affecting fertility can be involved with a number of male- and female-specific processes, or functional biological classifications, such as those shown in FIGS. 1-3. As shown in FIG. 1, female reproductive/fertility related processes, or classifications, include gonadogenesis, neuroendocrine axis, folliculogensis, oogenesis, oocyte-embryo transition, placentation, post-implantation development, adiposity, (female) reproductive anatomy, immune response, fertilization and other processes. Male reproductive/fertility related processes, or classifications, include gonadogenesis neuroendocrine axis, post-implantation development, adiposity, (male) reproductive anatomy, immune response, spermatogenesis, sperm maturation and capacitation, fertilization, mitosis, meiosis, spermiogenesis, and other processes, as shown in FIGS. 2 and 3. These processes are described in more detail below.

Gonadogenesis encompasses the processes regulating the development of the ovaries and testes, and involves, but is not limited to, primordial germ cell specification and proliferation. The neuroendocrine axis encompasses for example the physiological pathways and structures regulating the production and activity of hormones in a number of different tissues in the human body, including the brain and gonads. Folliculogenesis encompasses the physiological mechanisms regulating the development of primordial follicles to cystic follicles in the ovary. Oogenesis encompasses the physiological mechanisms regulating the development of primordial oocytes to mature meiosis-II stage oocytes ready to be fertilized, hence those that are specific to female reproductive biology. Oocyte-embryo transition encompasses the physiological mechanisms regulating the development of the early embryo and includes mechanisms related to egg quality, such as oocyte cytoplasmic lattice formation, and paternal effect mechanisms. Placentation (Embryonic) encompasses the embryo-specific physiological mechanisms regulating implantation and the development of the placenta. Placentation (Uterine) encompasses the uterus-specific physiological mechanisms regulating embryo implantation and the development of the placenta. Post-implantation development encompasses the physiological mechanisms regulating post-implantation embryo development, particularly those whose disruption might lead to abnormal development or pregnancy loss in humans. Adiposity encompasses the physiological mechanisms regulating adipose tissue and body weight, which are known to play an important, indirect role in mammalian fecundity and infertility. Reproductive anatomy encompasses any phenotype relating to anatomical changes that could impact reproduction, fecundity or fertility. Immune response encompasses phenotypes that are specific to aspects of immune response mechanisms, which are known to play an important role in mammalian reproduction and fertility.

Spermatogenesis encompasses the processes involved in the production or development of mature spermatozoa, hence those that are specific to male reproductive biology. Maturation encompasses processes that enable spermatozoa to fertilize eggs, hence those that are specific to male reproductive biology. Capacitation encompasses processes specific to functional capacitation of spermatozoa in the vaginal canal and uterus. Fertilization encompasses processes relating to the union of a human egg and sperm. Mitosis encompasses processes involving changes to the cell division process such that it does not end with two daughter cells that have the same chromosomal complement as the parent cell. Such changes to the mitotic process may affect for example fertility-related cell proliferation or tissue maintenance. Meiosis encompasses processes regulating meiosis such that it results in four daughter cells each with exactly half the chromosome complement of the parent cell, for example during gametogenesis. Spermiogenesis encompasses processes regulating the morphological differentiation of haploid cells into sperm.

Mutations in genes associated with these various processes result in fertility difficulties for males and/or females containing these mutations.

### Obtaining Genetic Data

Genetic data can be obtained, for example, by conducting an assay on a sample from a male or female that detects either a variant in a reproductive health-associated genetic region or abnormal (over or under) expression of a reproductive health-associated genetic region. The presence of certain variants in those genetic regions or abnormal expression levels of those genetic regions is indicative of fertility outcomes, i.e., whether ongoing pregnancy or live birth is achievable. Exemplary variants include, but are not limited to, a single nucleotide polymorphism, a single nucleotide variant, a deletion, an insertion, an inversion, a genetic rearrangement, a copy number variation, chromosomal microdeletion, genetic mosaicism, karyotype abnormality, or a combination thereof.

A sample may include a human tissue or bodily fluid and may be collected in any clinically acceptable manner. A tissue is a mass of connected cells and/or extracellular matrix material, e.g. skin tissue, hair, nails, nasal passage tissue, CNS tissue, neural tissue, eye tissue, liver tissue, kidney tissue, placental tissue, mammary gland tissue, gastrointestinal tissue, musculoskeletal tissue, genitourinary tissue, bone marrow, and the like, derived from, for example, a human or other mammal and includes the connecting material and the liquid material in association with the cells and/or tissues. A body fluid is a liquid material derived from, for example, a human or other mammal. Such body fluids include, but are not limited to, mucous, blood, plasma, serum, serum derivatives, bile, blood, maternal blood, phlegm, saliva, sputum, sweat, amniotic fluid, menstrual fluid, mammary fluid, follicular fluid of the ovary, fallopian tube fluid, peritoneal fluid, urine, semen, and cerebrospinal fluid (CSF), such as lumbar or ventricular CSF. A sample may also be a fine needle aspirate or biopsied tissue, e.g. an endometrial aspirate, breast tissue biopsy, and the like. A sample also may be media containing cells or biological material. A sample may also be a blood clot, for example, a blood clot that has been obtained from whole blood after the serum has been removed. In certain embodiments, the sample may include reproductive cells or tissues, such as gametic cells, gonadal tissue, fertilized embryos, and placenta. In certain embodiments, the sample is blood, saliva, or semen collected from the subject.

Genetic information from the sample can be obtained by nucleic acid extraction from the sample. Methods for extracting nucleic acid from a sample are known in the art. See for example, Maniatis, et al., Molecular Cloning: A Laboratory Manual, Cold Spring Harbor, N.Y., pp. 280-281, 1982, the contents of which are incorporated by reference herein in their entirety. In certain embodiments, a sample is collected from a subject followed by enrichment for genes or gene fragments of interest, for example by hybridization to a nucleotide array including fertility-related genetic regions or genetic fragments of interest. The sample may be enriched for genetic regions of interest (e.g., reproductive health-associated genetic regions) using methods known in the art, such as hybrid capture. See for examples, Lapidus (U.S. Pat. No. 7,666,593), the content of which is incorporated by reference herein in its entirety.

In particular embodiments, the assay is conducted on fertility-related genes or genetic regions containing the gene or a part thereof, such as those genes found in Table 3. Detailed descriptions of conventional methods, such as those employed to make and use nucleic acid arrays, amplification primers, hybridization probes, and the like can be found in standard laboratory manuals such as: Genome Analysis: A Laboratory Manual Series (Vols. I-IV), Cold Spring Harbor Laboratory Press; PCR Primer: A Laboratory Manual, Cold Spring Harbor Laboratory Press; and Sambrook, J et al., (2001) Molecular Cloning: A Laboratory Manual, 2nd ed. (Vols. 1-3), Cold Spring Harbor Laboratory Press. Custom nucleic acid arrays are commercially available from, e.g., Affymetrix (Santa Clara, Calif.), Applied Biosystems (Foster City, Calif.), and Agilent Technologies (Santa Clara, Calif.).

Methods of detecting variations (e.g., mutations) are known in the art. In certain embodiments, a known single nucleotide polymorphism (SNP) at a particular position can be detected by single base extension for a primer that binds to the sample DNA adjacent to that position. See for example Shuber et al. (U.S. Pat. No. 6,566,101), the content of which is incorporated by reference herein in its entirety. In other embodiments, a hybridization probe might be employed that overlaps the SNP of interest and selectively hybridizes to sample nucleic acids containing a particular nucleotide at that position. See for example Shuber et al. (U.S. Pat. Nos. 6,214,558 and 6,300,077), the content of which is incorporated by reference herein in its entirety.

In particular embodiments, nucleic acids are sequenced in order to detect variants in the nucleic acid compared to wild-type and/or non-mutated forms of the sequence. The nucleic acid can include a plurality of nucleic acids derived from a plurality of genetic elements. Methods of detecting sequence variants are known in the art, and sequence variants can be detected by any sequencing method known in the art.

DNA sequencing techniques include classic dideoxy sequencing reactions (Sanger method) using labeled terminators or primers and gel separation in slab or capillary, sequencing by synthesis using reversibly terminated labeled nucleotides, pyrosequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLiD sequencing. Sequencing of separated molecules has more recently been demonstrated by sequential or single extension reactions using polymerases or ligases as well as by single or sequential differential hybridizations with libraries of probes. Exemplary sequencing methods include but are not limited to the following: sequencing by chain termination and gel separation, as described by Sanger et al., Proc Natl. Acad. Sci. USA, 74(12): 5463 67 (1977); chemical degradation of nucleic acid fragments. See, Maxam et al., Proc. Natl. Acad. Sci., 74: 560 564 (1977); sequencing by hybridization. See, e.g., Harris et al., (U.S. patent application number 2009/0156412); Helicos True Single Molecule Sequencing (tSMS). See Harris T. D. et al. (2008) Science 320:106-109; see also, e.g., Lapidus et al. (U.S. Pat. No. 7,169,560), Lapidus et al. (U.S. patent application number 2009/0191565), Quake et al. (U.S. Pat. No. 6,818,395), Harris (U.S. Pat. No. 7,282,337), Quake et al. (U.S. patent application number 2002/0164629), and Braslaysky, et al., PNAS (USA), 100: 3960-3964 (2003); 454 sequencing (Roche) (Margulies, M et al. 2005, Nature, 437, 376-380); SOLiD technology (Applied Biosystems); Ion Torrent sequencing (U.S. patent application numbers 2009/0026082, 2009/0127589, 2010/0035252, 2010/0137143, 2010/0188073, 2010/0197507, 2010/0282617, 2010/0300559), 2010/0300895, 2010/0301398, and 2010/0304982); single molecule, real-time (SMRT) technology of Pacific Biosciences; nanopore sequencing (Soni G V and Meller A. (2007) Clin Chem 53: 1996-2001); chemical-sensitive field effect transistor (chemFET) arrays (See e.g., U.S. Patent Application Publication No. 2009/0026082); and use of an electron microscope (Moudrianakis E. N. and Beer M. Proc Natl Acad Sci USA. 1965 March; 53:564-71), the content of each of which is incorporated by reference herein in its entirety.

Yet another example of a sequencing technology that can be used in the methods of the provided invention is next-generation sequencing, such as Illumina sequencing, using Illumina HiSeq sequencers. Illumina sequencing is based on the amplification of DNA on a solid surface using fold-back PCR and anchored primers. Genomic DNA is fragmented, and adapters are added to the 5′ and 3′ ends of the fragments. DNA fragments that are attached to the surface of flow cell channels are extended and bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labeled, reversibly terminating nucleotides are used to perform sequential sequencing. After nucleotide incorporation, a laser is used to excite the fluorophores, and an image is captured and the identity of the first base is recorded. The 3′ terminators and fluorophores from each incorporated base are removed and the incorporation, detection and identification steps are repeated.

In certain aspects, the invention provides a microarray including a plurality of oligonucleotides attached to a substrate at discrete addressable positions, in which at least one of the oligonucleotides hybridizes to a portion of a gene suspected of affecting fertility in a man or woman. Methods of constructing microarrays are known in the art. See for example Yeatman et al. (U.S. patent application number 2006/0195269), the content of which is hereby incorporated by reference in its entirety.

If the nucleic acid from the sample is degraded or only a minimal amount of nucleic acid can be obtained from the sample, PCR can be performed on the nucleic acid in order to obtain a sufficient amount of nucleic acid for sequencing (See e.g., Mullis et al. U.S. Pat. No. 4,683,195, the contents of which are incorporated by reference herein in its entirety).

Sequencing by any of the methods described above and known in the art produces sequence reads. Sequence reads can be analyzed to call variants by any number of methods known in the art. Variant calling can include aligning sequence reads to a reference (e.g. hg18) and reporting single nucleotide polymorphism (SNP)/single nucleotide variant alleles. An example of methods for analyzing sequence reads and calling variants includes standard Genome Analysis Toolkit (GATK) methods. See The Genome Analysis Toolkit: a MapReduce framework for analyzing next-generation DNA sequencing data, Genome Res 20(9):1297-1303, the contents of each of which are incorporated by reference. GATK is a software package for analysis of high-throughput sequencing data capable of identifying variants, including SNPs. SNP/SNV alleles can be reported in a format such as a Sequence Alignment Map (SAM) or a Variant Call Format (VCF) file. Some background may be found in Li & Durbin, 2009, Fast and accurate short read alignment with Burrows-Wheeler Transform. Bioinformatics 25:1754-60 and McKenna et al., 2010. Variant calling produces results (“variant calls”) that may be stored as a sequence alignment map (SAM) or binary alignment map (BAM) file—comprising an alignment string (the SAM format is described, e.g., in Li, et al., The Sequence Alignment/Map format and SAMtools, Bioinformatics, 2009, 25(16):2078-9). Additionally or alternatively, output from the variant calling may be provided in a variant call format (VCF) file, e.g., in report. A typical VCF file will include a header section and a data section. The header contains an arbitrary number of meta-information lines, each starting with characters ‘##’, and a TAB delimited field definition line starting with a single ‘#’ character. The field definition line names eight mandatory columns and the body section contains lines of data populating the columns defined by the field definition line. The VCF format is described in Danecek et al., 2011, The variant call format and VCFtools, Bioinformatics 27(15):2156-2158. Further discussion may be found in U.S. Pub. 2013/0073214; U.S. Pub. 2013/0345066; U.S. Pub. 2013/0311106; U.S. Pub. 2013/0059740; U.S. Pub. 2012/0157322; U.S. Pub. 2015/0057946 and U.S. Pub. 2015/0056613, each incorporated by reference.

Furthermore, methods of the invention include conducting an assay on a sample from a subject that detects an abnormal (over or under) expression of a reproductive health-associated gene (e.g. a differentially or abnormally expressed gene). A differentially or abnormally expressed gene refers to a gene whose expression is activated to a higher or lower level in a subject suffering from a disorder, such as infertility, relative to its expression in a normal or control subject. The terms also include genes whose expression is activated to a higher or lower level at different stages of the same disorder. It is also understood that a differentially expressed gene may be either activated or inhibited at the nucleic acid level or protein level, or may be subject to alternative splicing to result in a different polypeptide product. Such differences may be evidenced by a change in mRNA levels, surface expression, secretion or other partitioning of a polypeptide, for example.

Differential gene expression may include a comparison of expression between two or more genes or their gene products, or a comparison of the ratios of the expression between two or more genes or their gene products, or even a comparison of two differently processed products of the same gene, which differ between normal subjects and subjects suffering from a disorder, such as infertility, or between various stages of the same disorder. Differential expression includes both quantitative, as well as qualitative, differences in the temporal or cellular expression pattern in a gene or its expression products. Differential gene expression (increases and decreases in expression) is based upon percent or fold changes over expression in normal cells. Increases may be of 1, 5, 10, 20, 30, 40, 50, 60, 70, 80, 90, 100, 120, 140, 160, 180, or 200% relative to expression levels in normal cells. Alternatively, fold increases may be of 1, 1.5, 2, 2.5, 3, 3.5, 4, 4.5, 5, 5.5, 6, 6.5, 7, 7.5, 8, 8.5, 9, 9.5, or 10 fold over expression levels in normal cells. Decreases may be of 1, 5, 10, 20, 30, 40, 50, 55, 60, 65, 70, 75, 80, 82, 84, 86, 88, 90, 92, 94, 96, 98, 99 or 100% relative to expression levels in normal cells.

Methods of detecting levels of gene products (e.g., RNA or protein) are known in the art. Commonly used methods known in the art for the quantification of mRNA expression in a sample include northern blotting and in situ hybridization (Parker & Barnes, Methods in Molecular Biology 106:247 283 (1999); RNAse protection assays (Hod, Biotechniques 13:852 854 (1992); and PCR-based methods, such as reverse transcription polymerase chain reaction (RT-PCR) (Weis et al., Trends in Genetics 8:263 264 (1992); the contents of all of which are incorporated by reference herein in their entirety. Alternatively, antibodies may be employed that can recognize specific duplexes, including RNA duplexes, DNA-RNA hybrid duplexes, or DNA-protein duplexes. Other methods known in the art for measuring gene expression (e.g., RNA or protein amounts) are shown in Yeatman et al. (U.S. patent application number 2006/0195269), the content of which is hereby incorporated by reference in its entirety.

In certain embodiments, reverse transcriptase PCR (RT-PCR) is used to measure gene expression. RT-PCR is a quantitative method that can be used to compare mRNA levels in different sample populations to characterize patterns of gene expression, to discriminate between closely related mRNAs, and to analyze RNA structure. Various methods are well known in the art. See, e.g., Ausubel et al., Current Protocols of Molecular Biology, John Wiley and Sons (1997); Rupp and Locker, Lab Invest. 56:A67 (1987), and De Andres et al., BioTechniques 18:42044 (1995); Held et al., Genome Research 6:986 994 (1996), the contents of which are incorporated by reference herein in their entirety.

Further PCR-based techniques include, for example, differential display (Liang and Pardee, Science 257:967 971 (1992)); amplified fragment length polymorphism (iAFLP) (Kawamoto et al., Genome Res. 12:1305 1312 (1999)); BeadArray™ technology (Illumina, San Diego, Calif.; Oliphant et al., Discovery of Markers for Disease (Supplement to Biotechniques), June 2002; Ferguson et al., Analytical Chemistry 72:5618 (2000)); BeadsArray for Detection of Gene Expression (BADGE), using the commercially available Luminex100 LabMAP system and multiple color-coded microspheres (Luminex Corp., Austin, Tex.) in a rapid assay for gene expression (Yang et al., Genome Res. 11:1888 1898 (2001)); and high coverage expression profiling (HiCEP) analysis (Fukumura et al., Nucl. Acids. Res. 31(16) e94 (2003)). The contents of each of which are incorporated by reference herein in their entirety.

In another embodiment, a MassARRAY-based gene expression profiling method is used to measure gene expression. For further details see, e.g. Ding and Cantor, Proc. Natl. Acad. Sci. USA 100:3059 3064 (2003), incorporated herein by reference.

In certain embodiments, differential gene expression can also be identified, or confirmed using a microarray technique. In this method, polynucleotide sequences of interest (including cDNAs and oligonucleotides) are plated, or arrayed, on a microchip substrate. The arrayed sequences are then hybridized with specific DNA probes from cells or tissues of interest. Methods for making microarrays and determining gene product expression (e.g., RNA or protein) are shown in Yeatman et al. (U.S. patent application number 2006/0195269); see also Schena et al., Proc. Natl. Acad. Sci. USA 93(2):106 149 (1996), the content of each of which is incorporated by reference herein in their entirety. Microarray analysis can be performed by commercially available equipment, following manufacturer's protocols, such as by using the Affymetrix GenChip technology, or Incyte's microarray technology.

In another aspect, protein levels can be determined by constructing an antibody microarray in which binding sites comprise immobilized, preferably monoclonal, antibodies specific to a plurality of protein species encoded by the cell genome. Methods for making monoclonal antibodies are well known (see, e.g., Harlow and Lane, 1988, ANTIBODIES: A LABORATORY MANUAL, Cold Spring Harbor, N.Y., which is incorporated in its entirety for all purposes).

In yet another aspect, levels of transcripts of marker genes in a number of tissue specimens may be characterized using a “tissue array” (Kononen et al., Nat. Med 4(7):844-7 (1998)). In other embodiments, Serial Analysis of Gene Expression (SAGE) is used to measure gene expression. Serial analysis of gene expression (SAGE) is a method that allows the simultaneous and quantitative analysis of a large number of gene transcripts, without the need of providing an individual hybridization probe for each transcript. For more details see, e.g. Velculescu et al., Science 270:484 487 (1995); and Velculescu et al., Cell 88:243 51 (1997, the contents of each of which are incorporated by reference herein in their entirety).

In other embodiments, Massively Parallel Signature Sequencing (MPSS) is used to measure gene expression. For more details see, e.g. Brenner et al., Nature Biotechnology 18:630 634 (2000).

Immunohistochemistry methods are also suitable for detecting the expression levels of the gene products of the present invention. In these methods, antibodies (monoclonal or polyclonal) or antisera, such as polyclonal antisera, specific for each marker are used to detect expression. Immunohistochemistry protocols and kits are well known in the art and are commercially available.

In certain embodiments, a proteomics approach is used to measure gene expression. Proteomics typically includes the following steps: (1) separation of individual proteins in a sample by 2-D gel electrophoresis (2-D PAGE); (2) identification of the individual proteins recovered from the gel, e.g. my mass spectrometry or N-terminal sequencing, and (3) analysis of the data using bioinformatics. Proteomics methods are valuable supplements to other methods of gene expression profiling, and can be used, alone or in combination with other methods, to detect the products of the prognostic markers of the present invention.

In some embodiments, mass spectrometry (MS) analysis can be used alone or in combination with other methods (e.g., immunoassays or RNA measuring assays) to determine the presence and/or quantity of the one or more biomarkers disclosed herein in a biological sample. In some embodiments, the MS analysis includes matrix-assisted laser desorption/ionization (MALDI) time-of-flight (TOF) MS analysis, such as for example direct-spot MALDI-TOF or liquid chromatography MALDI-TOF mass spectrometry analysis. In some embodiments, the MS analysis comprises electrospray ionization (ESI) MS, such as for example liquid chromatography (LC) ESI-MS. Mass analysis can be accomplished using commercially-available spectrometers. Methods for utilizing MS analysis, including MALDI-TOF MS and ESI-MS, to detect the presence and quantity of biomarker peptides in biological samples are known in the art. See, for example, U.S. Pat. Nos. 6,925,389; 6,989,100; and 6,890,763, each of which is incorporated by reference herein in their entirety.

### Methodologies for Assessing Probability of Achieving Ongoing Pregnancy

The present invention provides methods for generating a predicted probability of achieving ongoing pregnancy in an individual with respect to a specific fertility treatment and informing course of treatment therefrom, wherein the method specifically incorporates the effect of BMI on the predicted probability. Methods for generating a likelihood of achieving ongoing pregnancy generally involve the determination of one or more correlations between clinical characteristics and known pregnancy and infertility-related outcomes from a reference set of data to provide a model representative of a probability of ongoing pregnancy. In certain aspects, the methods further involve the determination of one or more correlations between genetic characteristics and known pregnancy and infertility-related outcomes from the reference set of data to adjust the model. The model can then be applied to the input data to generate the likelihood of achieving ongoing pregnancy in the subject, which will in turn, inform the course of treatment for the subject.

Clinical characteristics obtained from the reference population include, but are not limited to, any or all of the characteristics described above in the “Clinical Characteristics” section. Exemplary characteristics include BMI, fertility treatment history, age, BAFC, sperm motility, clinical diagnoses, and medication type. With respect to fertility treatment history, the reference set of data includes information as to what fertility treatments, including any ovulation induction agents, were used. Exemplary fertility treatments include, but are not limited to, ART, non-ART fertility treatments (RE), and fertility preservation technologies (egg, embryo, or ovarian preservation). Exemplary assisted reproductive technologies include, without limitation, IVF, zygote intrafallopian transfer (ZIFT), gametic intrafallopian transfer (GIFT), or ICSI paired with one of the methods above. Exemplary non-ART fertility treatments include ovulation induction protocols with or without IUI with sperm. Exemplary ovulation induction agents include gonadotropins such as LH, FSH, human menopausal gonadotropin (hMG), and hCG; and oral ovulation induction agents. Exemplary oral ovulation induction agents include, but are not limited to: clomiphene citrate; aromatase inhibitors, such as letrozole and anastrozole; insulin sensitizing drugs, such as metformin, rosiglitazone, and pioglitazone; bromocriptine; cabergoline; GnRH; and GnRH analogs, such as leuprolide acetate, nafarelin acetate, goserelin acetate, ganirelix, and cetrorelix acetate (the former three being agonists and the latter two being antagonists); and combinations thereof. Preferably, the clinical characteristics are to include BMI and previous fertility treatments, wherein at least some of the reference population has undergone non-ART treatments, such as IUI, with and without ovulation induction agents.

The clinical characteristics obtained from the reference population can then be passed through an association analysis in order to determine whether and to what extent the characteristics obtained from the subjects in the reference population are associated with the cumulative odds of achieving ongoing pregnancy.

In one embodiment, the methods also incorporate genetic characteristics from the reference population and their impact on the cumulative probability of achieving ongoing pregnancy. First, variants within genes and genetic regions, including those described above, are identified. In a preferred embodiment, whole genome sequencing is conducted on DNA extracted from whole blood samples using the Illumina HiSeq platform. As described above, variants can be called using standard Genome Analysis Toolkit (GATK) methods.

Once the variants are called, a customized pipeline is used to identify deleterious variants among the genetic signatures of patients. Deleterious variants can be determined using, for example, the SnpEff and Variant Effect Predictor (www.ensembl.org) engines. SnpEff is capable of rapidly categorizing the effects of SNPs and other variants in whole genome sequences. See, Cingolani et al., A program for annotating and predicting the effects of single nucleotide polymorphisms, SnpEff: SNPs in the genome of Drosophila melanogaster strain w¹¹¹⁸; iso-2; iso-3; Landes Bioscience, 6:2, 1-13; April/May/June 2012, incorporated herein by reference. Variants predicted to have a high impact or be ‘moderate missense variants’ (moderate is defined by SnpEff as causing an amino acid change) using programs such as SnpEff are then selected.

Upon identification of these high and moderate impact variants, the variants are then passed through a scoring system based on various annotation tools. One of ordinary skill in the art would understand that both molecular and computational approaches are available for annotating variants (e.g. by comparing to a known database, through the use of ANOVA technology, through the use of multivariant analysis). Exemplary annotation tools include the Database for Annotation, Visualization and Integrated Discover (DAVID). Nature Protocols 2009; 4(1):44; and Nucleic Acids Res. 2009; 37(1):1, incorporated herein by reference.

Variants that were considered deleterious by at least two annotation tools can then be passed through to the association analysis, along with the clinical characteristics to determine whether the genetic variant signatures obtained from the subjects are associated with their cumulative odds of ongoing pregnancy.

The association analysis involves the use of any one of a number of models to calculate cumulative odds of ongoing pregnancy for the reference population, such as a cohort of patients. As noted above, the model incorporates and adjusts for clinical information, such as the clinical characteristics listed in Table 1, obtained from the subjects, including BMI, age, BAFC, medication type, sperm motility, clinical diagnoses, and prior fertility treatments. In one aspect, the model can be weighted towards more recent data.

Suitable analysis methods include, without limitation, logistic regression, ordinal logistic regression, linear or quadratic discriminant analysis, clustering, principal component analysis, nearest neighbor classifier analysis, and discrete time-proportional hazards models.

Logistic regression analysis may be used to generate an odds ratio and relative risk for each characteristic. Method of logistic regression are described, for example in, Ruczinski (Journal of Computational and Graphical Statistics 12:475-512, 2003); Agresti (An Introduction to Categorical Data Analysis, John Wiley & Sons, Inc., 1996, New York, Chapter 8); and Yeatman et al. (U.S. patent application number 2006/0195269), the content of each of which is hereby incorporated by reference in its entirety.

Some embodiments of the present invention provide generalizations of the logistic regression model that handle multicategory (polychotomous) responses. Such embodiments can be used to discriminate an organism into one or more prognosis groups (e.g., good prognosis, poor prognosis). Such regression models use multicategory logit models that simultaneously refer to all pairs of categories, and describe the odds of response in one category instead of another. Once the model specifies logits for a certain (J-1) pairs of categories, the rest are redundant. See, for example, Agresti, An Introduction to Categorical Data Analysis, John Wiley & Sons, Inc., 1996, New York, Chapter 8, which is hereby incorporated by reference.

Regularization techniques may be used in certain embodiments of the invention in order to prevent over-fitting to training data and to identify the most important features to include in predictive models. Examples of regularization techniques include, without limitation, least absolute shrinkage and selection operator (lasso), ridge regression, elastic net, or certain specifications of hierarchical Bayesian models. Regularization can be used with any known algorithm which optimizes an objective function including, without limitation, linear regression, logistic regression, or artificial neural networks. See, for example, Chapter 16 of Hastie, 2001, The Elements of Statistical Learning, Springer, New York, hereby incorporated by reference.

Lasso regularization results in penalized parameter estimates which are smaller in magnitude than non-penalized estimates. Lasso regression can be used as a variable selection technique by driving parameter estimates exactly to 0, suggesting that these features do not have predictive ability in the model. Only features with parameter estimates larger than 0 after lasso regularization are then taken as features in predictive models.

Linear discriminant analysis (LDA) attempts to classify a subject into one of two categories based on certain object properties. In other words, LDA tests whether object attributes measured in an experiment predict categorization of the objects. LDA typically requires continuous independent variables and a dichotomous categorical dependent variable. In one embodiment, the selected fertility-associated phenotypic traits serve as the requisite continuous independent variables. The prognosis group classification of each of the members of the training population serves as the dichotomous categorical dependent variable. For more information on linear discriminant analysis, see Duda, Pattern Classification, Second Edition, 2001, John Wiley & Sons, Inc; and Hastie, 2001, The Elements of Statistical Learning, Springer, New York; Venables & Ripley, 1997, Modern Applied Statistics with s-plus, Springer, New York, incorporated herein by reference.

Quadratic discriminant analysis (QDA) takes the same input parameters and returns the same results as LDA. QDA uses quadratic equations, rather than linear equations, to produce results. LDA and QDA are interchangeable, and which to use is a matter of preference and/or availability of software to support the analysis. Logistic regression takes the same input parameters and returns the same results as LDA and QDA.

In some embodiments of the present invention, decision trees are used to classify patients using expression data for a selected set of molecular markers of the invention. Decision tree algorithms belong to the class of supervised learning algorithms. The aim of a decision tree is to induce a classifier (a tree) from real-world example data. This tree can be used to classify unseen examples which have not been used to derive the decision tree. In general there are a number of different decision tree algorithms, many of which are described in Duda, Pattern Classification, Second Edition, 2001, John Wiley & Sons, Inc. Decision tree algorithms often require consideration of feature processing, impurity measure, stopping criterion, and pruning. Specific decision tree algorithms include, but are not limited to classification and regression trees (CART), multivariate decision trees, ID3, and C4.5.

In some embodiments, the fertility-associated characteristics are used to cluster a training set. Additional information and examples are described in Duda and Hart, Pattern Classification and Scene Analysis, 1973, John Wiley & Sons, Inc., New York; Kaufman and Rousseeuw, 1990, Finding Groups in Data: An Introduction to Cluster Analysis, Wiley, New York, N.Y.; Duda, Pattern Classification, Second Edition, 2001, John Wiley & Sons, Inc; and Hastie, 2001, The Elements of Statistical Learning, Springer, New York; Everitt, 1993, Cluster analysis (3d ed.), Wiley, New York, N.Y.; and Backer, 1995, Computer-Assisted Reasoning in Cluster Analysis, Prentice Hall, Upper Saddle River, N.J. Particular exemplary clustering techniques that can be used in the present invention include, but are not limited to, hierarchical clustering (agglomerative clustering using nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering.

Other algorithms for analyzing associations are known. For example, the stochastic gradient boosting is used to generate multiple additive regression tree (MART) models to predict a range of outcome probabilities. A different approach called the generalized linear model, expresses the outcome as a weighted sum of functions of the predictor variables. The weights are calculated based on least squares or Bayesian methods to minimize the prediction error on the training set. A predictor's weight reveals the effect of changing that predictor, while holding the others constant, on the outcome. In cases where one or more predictors are highly correlated, in a phenomenon known as collinearity, the relative values of their weights are less meaningful; steps must be taken to remove that collinearity, such as by excluding the nearly redundant variables from the model. Thus, when properly interpreted, the weights express the relative importance of the predictors. Less general formulations of the generalized linear model include linear regression, multiple regression, and multifactor logistic regression models, and are highly used in the medical community as clinical predictors.

In a preferred embodiment, a discrete time-proportional odds model, such as the Cox proportional hazards model, is used to determine the cumulative probability of ongoing pregnancy in a group of subjects. See e.g., Cox, David R (1972). “Regression Models and Life-Tables”. Journal of the Royal Statistical Society, Series B. 34 (2): 187-220, incorporated herein by reference. Proportional hazards models relate the time that passes before some event occurs to one or more covariates that may be associated with that quantity of time, wherein the unique effect of a unit increase in a covariate is multiplicative with respect to the hazard rate (e.g., odds of achieving ongoing pregnancy/live birth).

To further enhance the predictive power of the analysis, as noted above, genetic information from the subjects can be incorporated. One method for determining the effect that genetic information has on the cumulative odds of ongoing pregnancy includes the sequence kernel association testing (SKAT) method, which is a gene set level methodology for testing if SNP-sets (gene sets) are associated with phenotypes (continuous or discrete) of interest. See Wu M C, Lee S, Cai T, Li Y, Boehnke M, Lin X. Rare-Variant Association Testing for Sequencing Data with the Sequence Kernel Association Test. American Journal of Human Genetics. 2011;89(1):82-93. doi:10.1016/j.ajhg.2011.05.029, incorporated herein by reference. For additional description of the incorporation of genetic factors into a model for assessing the probability of achieving ongoing pregnancy and specifically regarding the use of SKAT in adjusting the model, see U.S. Provisional Application No. 62/408,632, filed Oct. 14, 2016, incorporated herein by reference. Furthermore, burden testing can be used to enhance the results of the SKAT analysis given that SKAT only provides a P-value for evidence of an association between the SNP-set and phenotype of interest. Adjustment of models using SKAT-type analysis, allows one to see whether there is statistical evidence that genomic information, at the category level (e.g. functional biological classification level), provides additional information beyond known clinical metrics that is sufficient to significantly affect the model, and therefore be associated with the odds of achieving ongoing pregnancy.

Once the model has been developed based on a reference set of data which includes BMI and for which results of fertility treatments with or without ovulation induction agents are known, the model can be applied to data obtained from a patient suspected of having impaired fertility in order to predict the potential for achieving ongoing pregnancy with respect to a specific fertility treatment. The data obtained from the patient will include a determination of the patient's BMI. In this way, the patient can receive the predicted probabilities for achieving ongoing pregnancy using any number of fertility treatments, such as intrauterine insemination (IUI), in addition to whether one or more ovulation induction agents will affect the probability of achieving ongoing pregnancy. This information will inform the course of treatment for the individual.

Methods for Recommending Treatment and/or Treating a Patient

In one embodiment, methods of treatment or targeting treatment upon assessment of the patient's potential for achieving ongoing pregnancy are provided. As provided above, a patient's probability for achieving ongoing pregnancy can be determined with respect to a specific fertility treatment. Thus, the recommended treatment protocol will depend, in part, on the probability generated in accordance with the description above.

Exemplary fertility treatments include, but are not limited to, assisted reproductive technologies (ART), non-ART fertility treatments (RE), and fertility preservation technologies (egg, embryo, or ovarian preservation). Exemplary assisted reproductive technologies include, without limitation, in vitro fertilization (IVF), zygote intrafallopian transfer (ZIFT), gametic intrafallopian transfer (GIFT), or intracytoplasmic sperm injection (ICSI) paired with one of the methods above.

In IVF, eggs are removed from the female subject, fertilized outside the body, and implanted inside the uterus of the female subject. ZIFT is similar to IVF in that eggs are removed and fertilization of the eggs occurs outside the body. In ZIFT, however, the eggs are implanted in the Fallopian tube rather than the uterus. GIFT involves transferring eggs and sperm into the female subject's Fallopian tube. Accordingly, fertilization occurs inside the woman's body. In ICSI, a single sperm is injected into a mature egg that has removed from the body. The embryo is then transferred to the uterus or Fallopian tube. In RE, hormone stimulation is used to improve the woman's fertility. Exemplary fertility preservation treatments include egg freezing in which eggs are removed, vitrified or otherwise frozen, and then stored indefinitely. Preservation can similarly be achieved through cryo-preservation of embryos generated through IVF and cryo-preservation of ovarian tissue, including slices of the ovarian cortex. Preservation could also involve removal of the ovary from the pelvic region and subcutaneous implantation in an ectopic location such as under the skin the in periphery of the body (i.e. arm).

Exemplary non-ART fertility treatments include ovulation induction protocols with or without IUI with sperm. Exemplary ovulation induction agents include gonadotropins such as LH, FSH, hMG, and hCG; and oral ovulation induction agents. Exemplary oral ovulation induction agents include, but are not limited to: clomiphene citrate; aromatase inhibitors, such as letrozole and anastrozole; insulin sensitizing drugs, such as metformin, rosiglitazone, and pioglitazone; bromocriptine; cabergoline; GnRH; and GnRH analogs, such as leuprolide acetate, nafarelin acetate, goserelin acetate, ganirelix, and cetrorelix acetate (the former three being agonists and the latter two being antagonists); and combinations thereof.

In one embodiment, the fertility treatment protocol involves the use of ovulation induction agents, the effect of which on the probability of achieving ongoing pregnancy is dependent, in part, on the patient's BMI. A patient's BMI can influence the effectiveness of certain fertility treatments. For instance, the use of gonadotropins alone as ovulation induction agents prior to intrauterine insemination (IUI), regardless of BMI, are associated with a higher change of achieving ongoing pregnancy. However, the use of gonadotropins alone is associated with an increased risk for multiples. Additionally, the divergence in success rate increases as BMI increases, with the use of gonadotropins alone being associated with a higher chance of achieving ongoing pregnancy than the use of gonadotropins with oral ovulation induction agents. Thus, the treatment protocol for a given patient will vary with respect to the patient's BMI and other competing factors, such as the risk for multiples.

In one embodiment, the BMI of a patient and its effect on the chances for achieving ongoing pregnancy with respect to certain fertility treatment protocols, including those that involve ovulation induction agents, is built into the assessment of a patient's probability for achieving ongoing pregnancy, such that the probability of achieving ongoing pregnancy in a patient can be provided for both an ovulation induction protocol involving the use of one or more gonadotropins only and an ovulation induction protocol involving the use of one or more gonadotropins in addition to the use of one or more oral ovulation induction agents. In this way, depending on which protocol provides the highest probability of achieving ongoing pregnancy and to what extent the probabilities diverge, a treatment protocol can be recommended. For example, for those individuals having a BMI near or under a threshold BMI, for which BMI does not play as much of a role in affecting a patient's probability of achieving ongoing pregnancy, the weighting of factors such as the risk for higher multiples will play a role in determining whether to add an oral ovulation induction agent to the ovulation induction protocol. Whereas, for those individuals having a higher BMI, such as a BMI near or above a threshold BMI which impacts the patient's probability of achieving ongoing pregnancy, the ovulation induction protocol will only include the use of gonadotropins.

In another embodiment, methods of the invention involve the determination of a patient's BMI and recommending/administering a certain ovulation induction protocol depending on the BMI. The methods can involve recommending and/or administering a gonadotropin alone if the patient's BMI is near or above a threshold limit. If the patient's BMI is near or below the threshold limit, a gonadotropin and optionally an oral ovulation induction agent is recommended and/or administered, with recommendation of the oral ovulation induction agent based on a weighting of other factors, such as the risk for multiples. The threshold BMI limit can be 18.5, 20, 21, 22, 23, 24, 25, 26, 27, 28, 29, 30, 31, 32, 33, 34, 35, or higher. In one embodiment, the threshold limit is 20. In another embodiment, the threshold limit is 25. In yet another embodiment, the threshold limit is 30.

In one embodiment, the treatment involves the use of IUI with an ovulation induction protocol, wherein the protocol involves the use of one or more gonadotropins, and optionally the use of one or more oral ovulation induction agents depending, in part, on the BMI of the patient.

### Systems

Aspects of the invention described herein can be performed using any type of computing device, such as a computer, that includes a processor, e.g., a central processing unit, or any combination of computing devices where each device performs at least part of the process or method. In some embodiments, systems and methods described herein may be performed with a handheld device, e.g., a smart tablet, or a smart phone, or a specialty device produced for the system.

Methods of the invention can be performed using software, hardware, firmware, hardwiring, or combinations of any of these. Features implementing functions can also be physically located at various positions, including being distributed such that portions of functions are implemented at different physical locations (e.g., imaging apparatus in one room and host workstation in another, or in separate buildings, for example, with wireless or wired connections).

Processors suitable for the execution of computer program include, by way of example, both general and special purpose microprocessors, and any one or more processor of any kind of digital computer. Generally, a processor will receive instructions and data from a read-only memory or a random access memory or both. The essential elements of computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example semiconductor memory devices, (e.g., EPROM, EEPROM, solid state drive (SSD), and flash memory devices); magnetic disks, (e.g., internal hard disks or removable disks); magneto-optical disks; and optical disks (e.g., CD and DVD disks). The processor and the memory can be supplemented by, or incorporated in, special purpose logic circuitry.

To provide for interaction with a user, the subject matter described herein can be implemented on a computer having an I/O device, e.g., a CRT, LCD, LED, or projection device for displaying information to the user and an input or output device such as a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

The subject matter described herein can be implemented in a computing system that includes a back-end component (e.g., a data server), a middleware component (e.g., an application server), or a front-end component (e.g., a client computer having a graphical user interface or a web browser through which a user can interact with an implementation of the subject matter described herein), or any combination of such back-end, middleware, and front-end components. The components of the system can be interconnected through network by any form or medium of digital data communication, e.g., a communication network. For example, the reference set of data may be stored at a remote location and the computer communicates across a network to access the reference set to compare data derived from the female subject to the reference set. In other embodiments, however, the reference set is stored locally within the computer and the computer accesses the reference set within the CPU to compare subject data to the reference set. Examples of communication networks include cell network (e.g., 3G or 4G), a local area network (LAN), and a wide area network (WAN), e.g., the Internet.

The subject matter described herein can be implemented as one or more computer program products, such as one or more computer programs tangibly embodied in an information carrier (e.g., in a non-transitory computer-readable medium) for execution by, or to control the operation of, data processing apparatus (e.g., a programmable processor, a computer, or multiple computers). A computer program (also known as a program, software, software application, app, macro, or code) can be written in any form of programming language, including compiled or interpreted languages (e.g., C, C++, Perl), and it can be deployed in any form, including as a stand-alone program or as a module, component, subroutine, or other unit suitable for use in a computing environment. Systems and methods of the invention can include instructions written in any suitable programming language known in the art, including, without limitation, C, C++, Perl, Java, ActiveX, HTMLS, Visual Basic, or JavaScript.

A computer program does not necessarily correspond to a file. A program can be stored in a file or a portion of file that holds other programs or data, in a single file dedicated to the program in question, or in multiple coordinated files (e.g., files that store one or more modules, sub-programs, or portions of code). A computer program can be deployed to be executed on one computer or on multiple computers at one site or distributed across multiple sites and interconnected by a communication network.

A file can be a digital file, for example, stored on a hard drive, SSD, CD, or other tangible, non-transitory medium. A file can be sent from one device to another over a network (e.g., as packets being sent from a server to a client, for example, through a Network Interface Card, modem, wireless card, or similar).

Writing a file according to the invention involves transforming a tangible, non-transitory computer-readable medium, for example, by adding, removing, or rearranging particles (e.g., with a net charge or dipole moment into patterns of magnetization by read/write heads), the patterns then representing new collocations of information about objective physical phenomena desired by, and useful to, the user. In some embodiments, writing involves a physical transformation of material in tangible, non-transitory computer readable media (e.g., with certain optical properties so that optical read/write devices can then read the new and useful collocation of information, e.g., burning a CD-ROM). In some embodiments, writing a file includes transforming a physical flash memory apparatus such as NAND flash memory device and storing information by transforming physical elements in an array of memory cells made from floating-gate transistors. Methods of writing a file are well-known in the art and, for example, can be invoked manually or automatically by a program or by a save command from software or a write command from a programming language.

Suitable computing devices typically include mass memory, at least one graphical user interface, at least one display device, and typically include communication between devices. The mass memory illustrates a type of computer-readable media, namely computer storage media. Computer storage media may include volatile, nonvolatile, removable, and non-removable media implemented in any method or technology for storage of information, such as computer readable instructions, data structures, program modules, or other data. Examples of computer storage media include RAM, ROM, EEPROM, flash memory, or other memory technology, CD-ROM, digital versatile disks (DVD) or other optical storage, magnetic cassettes, magnetic tape, magnetic disk storage or other magnetic storage devices, radiofrequency identification tags or chips, or any other medium which can be used to store the desired information and which can be accessed by a computing device.

As one skilled in the art would recognize as necessary or best-suited for performance of the methods of the invention, a computer system or machines of the invention include one or more processors (e.g., a central processing unit (CPU) a graphics processing unit (GPU) or both), a main memory and a static memory, which communicate with each other via a bus.

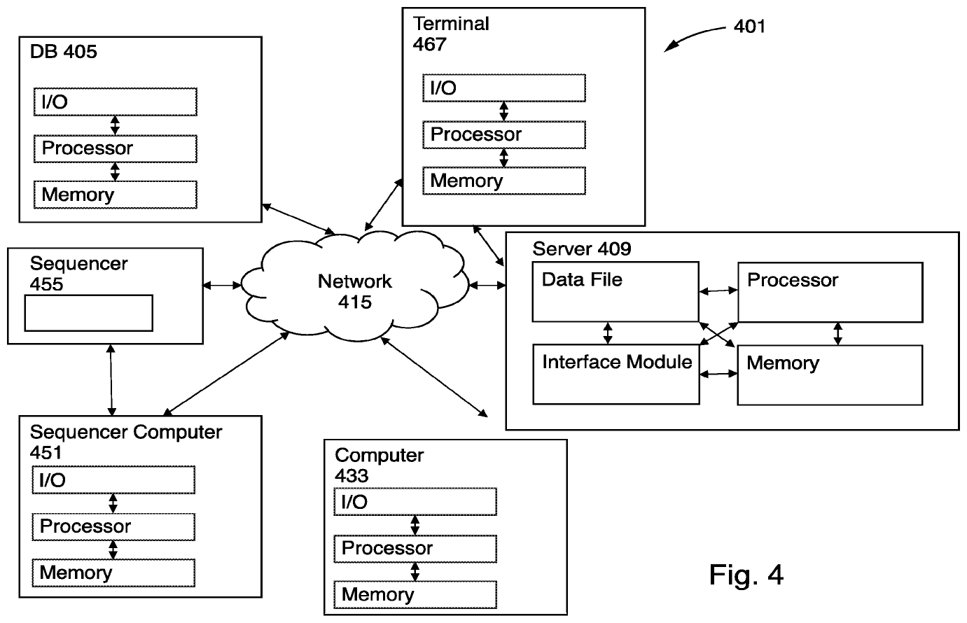

In an exemplary embodiment shown in FIG. 4, system 401 can include a computer 433 (e.g., laptop, desktop, or tablet). The computer 433 may be configured to communicate across a network 415. Computer 433 includes one or more processor and memory as well as an input/output mechanism. Where methods of the invention employ a client/server architecture, any steps of methods of the invention may be performed using server 409, which includes one or more of processor and memory, capable of obtaining data, instructions, etc., or providing results via interface module or providing results as a file. Server 409 may be engaged over network 415 through computer 433 or terminal 467, or server 415 may be directly connected to terminal 467, including one or more processor and memory, as well as input/output mechanism. In some embodiments, systems include an instrument 455 for obtaining sequencing data, which may be coupled to a sequencer computer 451 for initial processing of sequence reads

Memory according to the invention can include a machine-readable medium on which is stored one or more sets of instructions (e.g., software) embodying any one or more of the methodologies or functions described herein. The software may also reside, completely or at least partially, within the main memory and/or within the processor during execution thereof by the computer system, the main memory and the processor also constituting machine-readable media. The software may further be transmitted or received over a network via the network interface device.

Other embodiments are within the scope and spirit of the invention. For example, due to the nature of software, functions described above can be implemented using software, hardware, firmware, hardwiring, or combinations of any of these. Features implementing functions can also be physically located at various positions, including being distributed such that portions of functions are implemented at different physical locations.

### EXAMPLE 1

In this example, a multi-center retrospective analysis was performed and the effect of BMI on fertility treatment outcomes was examined.

### Study Design and Methodology

Between 2009 to 2015, 249,436 treatment cycles were analyzed from 113,809 patients undergoing timed intercourse (TI) with oral fertility medications (“orals”), IUI with/without orals and/or gonadotropin stimulation (Gnd), and IVF (excluding canceled cycles) were studied. Cox proportional hazards models with time-dependent covariates to model number of treatment cycles to ongoing pregnancy was utilized. Then regression was used to compare BAFC and

AMH across BMIs. We adjusted our models for age, BAFC, medication type, sperm motility, clinic, and diagnoses.

### Results

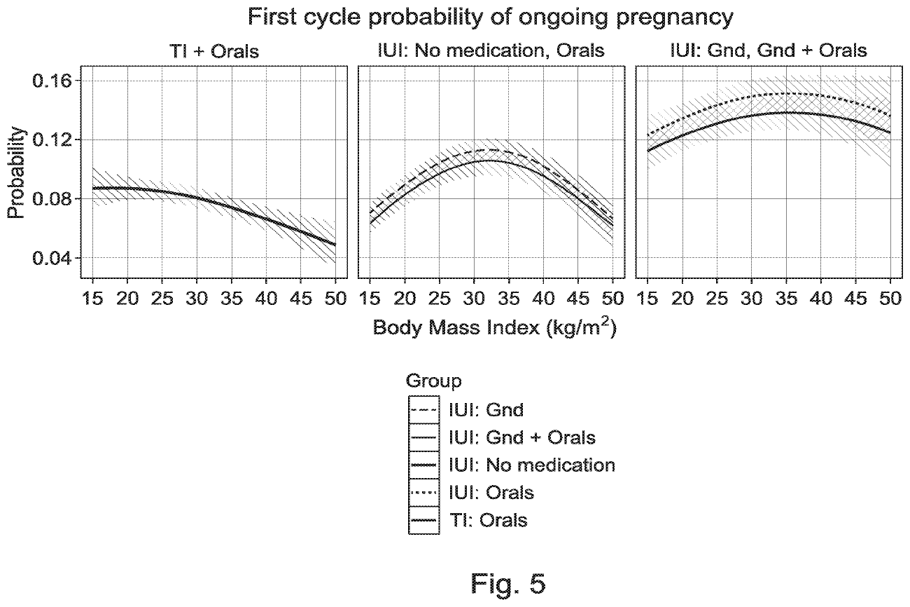

It was observed that women <18.5 BMI had reduced BAFC (P=0.02), but similar AMH levels to women with normal BMI (P=0.12). These results suggest that BMI was correlated with the probability of achieving ongoing pregnancy in a protocol-dependent fashion (FIG. 5, P<0.001). Interestingly, inflection points in success probabilities did not correspond with normal BMI boundaries. Surprisingly, underweight women undergoing IVF were at significantly increased odds of having cancelations or no embryos for transfer compared to women with normal BMI (OR=1.14, P=0.038, adjusting for age, BAFC). In Table 4 below, the inventors summarize the chances of achieving ongoing pregnancy in cycle 1 across BMI and cycle treatment type.

TABLE 4
Relationship between BMI and chances of achieving ongoing pregnancy, by
treatment type

| | | TI + Orals | IUI/IUI + Orals | IUI + Gnd | IVF |
|---|---|---|---|---|---|
| NIH Category | BMI | (n = 30,067) | (n = 93,974) | (n = 61,600) | (n = 63,795) |
| Underweight | <18.5 | 8.6% | 6.9% | 11.5% | 50.4% |
| Normal | 18.5-24.9 | 8.6% | 8.6% | 12.7% | 50.8% |
| Overweight | 25.0-29.9 | 8.3% | 10.0% | 13.6% | 50.0% |
| Obese class I | 30-34.9 | 7.8% | 10.4% | 14.1% | 48.3% |
| Obese class II | 35-39.9 | 7.1% | 10.0% | 14.2% | 45.5% |
| Obese class III | ≥40 | 5.8% | 7.8% | 13.5% | 39.3% |

These results suggest that normal BMI do not translate to optimal BMI for reproductive outcomes and success with fertility treatments.

### EXAMPLE 2

In this example, the influence of obesity and ovulation induction agents, specifically the effectiveness of ovulation induction with gonadotropins alone and with gonadotropins plus oral ovulation induction agents in relation to BMI was investigated.

### Study Design and Methodology

We analyzed 67,662 treatment cycles from 33,867 patients undergoing IUI ovulation induction (OI) with gonadotropins alone or with oral agents and gonadotropin from 2009 to 2016.

In this study, discrete-time proportional-odds models with time-dependent covariates to model the odds of achieving ongoing pregnancy during a given cycle were used. Interaction terms between BMI and stimulation type were included in the models to test the hypothesis that the effect of BMI was modified by stimulation type. BMI was treated continuously and categorized according to NIH guidelines (<18.5, 18.5-25, 25-30, 30-35, >35). Models were adjusted for age, BAFC, medication type, sperm motility, clinic, and diagnoses and were weighted towards more recent data.

### Results

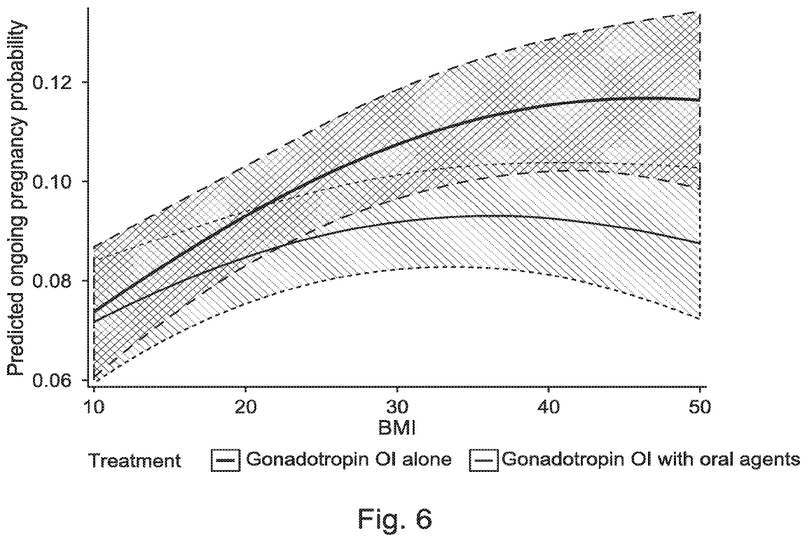

Overall, OI with only gonadotropin agents had significantly higher odds of achieving ongoing pregnancy compared to OI with oral agents and gonadotropins (odds ratio (OR)=1.16, p<0.05). However, we found that the impact of BMI on success rate differed significantly with the type of IUI treatment protocol. As shown in FIG. 6, the effect of BMI was nonlinear and peaked in the obesity categories of both protocols. At lower levels of BMI, there was no significant difference in the odds of having an ongoing pregnancy between stimulation types. However, there was a pronounced divergence in success rate as BMI increased, with gonadotropin OI alone performing better than gonadotropin OI with oral agents. When subcategories of BMI were assessed, a statistically significant difference in success was observed in women with a BMI of 25-30. Additionally, gonadotropin OI alone was associated with higher odds of multiples than gonadotropin with oral agents (OR=1.53, p<0.05), independent of patient BMI.

In sum, oral OI agents when used in conjunction with gonadotropins appear to be less effective for obese women. Although it had been previously demonstrated that oral agents alone result in lower rates of pregnancy with timed intercourse and IUI in obese women, the detrimental effect of increased BMI was not observed with gonadotropin alone. Surprisingly, we demonstrate that OI with only gonadotropin agents is associated with higher success rates than OI with oral agents and gonadotropins. This difference is most pronounced in higher BMI patients. Additionally, the study demonstrates that gonadotropin OI alone is related to a higher probability of conceiving multiples than oral and gonadotropin independent of BMI. These data suggest that the best IUI protocol for a given patient varies with respect to their BMI. As such, a personalized medicine approach including BMI should be taken into account when selecting an IUI treatment protocol.

### EXAMPLE 3

In this example, we performed a multi-center retrospective analysis and examined the effect of BMI on IVF cycle outcomes (cycle cancellation, oocytes retrieved, usable embryos, ongoing pregnancy) while controlling for confounding factors.

### Study Design and Methodology

We performed a retrospective review of 51,198 women who initiated their first autologous IVF or ICSI cycles at 13 fertility treatment centers in the United States between 2009-2015. We excluded frozen transfers of supernumerary embryos, cycles using preimplantation genetic screening (PGS), and cycles that were missing BMI data. We used BMI categories defined by World Health Organization (WHO): <18.50 kg/m² (underweight), 18.50-24.99 kg/m² (normal), 25.00-29.99 kg/m² (overweight), 30.00-34.99 kg/m² (obese class I), 35.00-39.99 kg/m² (obese class II), and ≥40.00 kg/m² (obese class III). Obese class II and class III were combined in our study because of limited sample size in obese class III group. The least absolute shrinkage and selection operator (LASSO) regression was used to select confounders that were statistically significantly (p<0.05) associated with each outcome measure. Logistic or Poisson regression was used to calculate the multivariate adjusted odds ratio (aOR; for cycle cancellation and ongoing clinical pregnancy) or adjusted incidence rate ratio (aIRR; for number of oocytes retrieved and number of usable embryos), respectively, with their 95% confidence interval (CI), with normal BMI as the reference category. Sensitivity analyses for number of usable embryos and ongoing clinical pregnancy were performed among cycles that transferred only blastocyst stage embryos.

### Results

We found BMI above the normal range was associated with worse IVF outcomes, including higher odds of cycle cancellation (cancelation of stimulation cycles that do not produce an adequate number of follicles to go through egg retrieval procedure), lower oocyte and embryo counts, and lower odds of an ongoing clinical pregnancy.

For cycle cancellation, factors that were controlled for in the analysis included: female age, gravidity, BAFC, basal FSH and E₂ levels, AMH, total gonadotropin used, infertility diagnosis, and clinic. After adjusting for these confounders, odds of cycle cancellation were comparable between underweight and normal weight patients (p=0.23). For patients with BMI≥18.5 kg/m², odds of cycle cancellation increased with rising BMI (overweight vs. normal weight aOR 1.17, 95% CI 1.08-1.26, p<0.001; obese class I vs. normal weight aOR 1.28, 95% CI 1.15-1.41, p<0.001), with obese class II/III patients having the highest aOR for cycle cancellation when compared with normal weight patients (aOR 1.50, 95% CI 1.33-1.68, p<0.001) (Table 5).

TABLE 5
The effect of BMI on IVF success

| | | | | N (%) of cases | | aOR or aIRR | |
|---|---|---|---|---|---|---|---|
| | BMI (kg/m²) categories | | Total N | or mean ± SD | | (95% CI) | P value |
| Cancellation | Underweight | <18.5 | 1377 | 125 | (9.1) | 1.13 (0.92, 1.39) | 0.23 |
| | Normal | 18.5-24.9 | 27945 | 2660 | (9.5) | 1.00 (reference) | |
| | Overweight | 25.0-29.9 | 12283 | 1356 | (11.0) | 1.17 (1.08, 1.26) | <0.001 |
| | Obese class I | 30.0-34.9 | 5791 | 648 | (11.2) | 1.28 (1.15, 1.41) | <0.001 |
| | Obese class II/III | ≥35.0 | 3802 | 459 | (12.1) | 1.50 (1.33, 1.68) | <0.001 |
| Number of oocytes | Underweight | <18.5 | 1252 | 14.3 ± 8.9 | | 0.99 (0.97, 1.00) | 0.11 |
| retrieved | Normal | 18.5-24.9 | 25285 | 14.1 ± 8.5 | | 1.00 (reference) | |
| | Overweight | 25.0-29.9 | 10927 | 13.9 ± 8.5 | | 1.00 (0.99, 1.00) | 0.19 |
| | Obese class I | 30.0-34.9 | 5143 | 14.0 ± 8.7 | | 0.98 (0.98, 0.99) | <0.001 |
| | Obese class II/III | ≥35.0 | 3343 | 13.2 ± 7.9 | | 0.93 (0.92, 0.94) | <0.001 |
| Number of usable | Underweight | <18.5 | 1249 | 3.6 ± 3.4 | | 0.95 (0.92, 0.97) | <0.001 |
| embryos | Normal | 18.5-24.9 | 25191 | 3.8 ± 3.6 | | 1.00 (reference) | |
| | Overweight | 25.0-29.9 | 10891 | 3.7 ± 3.4 | | 0.98 (0.97, 0.99) | 0.006 |
| | Obese class I | 30.0-34.9 | 5117 | 3.7 ± 3.4 | | 0.97 (0.96, 0.99) | <0.001 |
| | Obese class II/III | ≥35.0 | 3319 | 3.5 ± 3.3 | | 0.95 (0.93, 0.97) | <0.001 |
| Ongoing clinical | Underweight | <18.5 | 1011 | 465 | (46.0) | 0.92 (0.81, 1.05) | 0.21 |
| pregnancy | Normal | 18.5-24.9 | 21219 | 10050 | (47.4) | 1.00 (reference) | |
| | Overweight | 25.0-29.9 | 9411 | 4264 | (45.3) | 0.96 (0.91, 1.01) | 0.09 |
| | Obese class I | 30.0-34.9 | 4481 | 1959 | (43.7) | 0.89 (0.83, 0.95) | <0.001 |
| | Obese class II/III | ≥35.0 | 2933 | 1254 | (42.8) | 0.86 (0.79, 0.93) | <0.001 |

For number of oocytes retrieved, factors that were controlled for in the analysis included: female age, parity, BAFC, basal FSH, LH, and E₂ levels, AMH, total gonadotropin used, infertility diagnosis, and clinic. After adjusting for these confounders, among 45,950 patients that reached oocytes retrieval, obese patients had fewer oocytes retrieved compared to normal weight patients (Table 5). When comparing number of oocytes retrieved between obese class I and normal weight patients, aIRR was 0.98 (95% CI 0.98-0.99, p<0.001), while aIRR comparing obese class II/III with normal weight, was 0.93 (95% CI 0.92-0.94, p<0.001). Underweight and overweight patients had similar number of oocytes retrieved compared to normal weight patients (p=0.11 and p=0.19 respectively).

For number of usable embryos, factors that were controlled for in the analysis included: female age, BAFC, basal FSH, LH, and E₂ levels, AMH, gravidity, parity, ICSI, number of oocytes retrieved, embryo stage at the end of culture, total gonadotropin used, infertility diagnosis, and clinic. After adjusting for these confounders, among 45,767 patients with one or more oocytes retrieved, underweight, overweight, and obese patients had fewer usable embryos that were transferred or cryopreserved (underweight vs. normal weight aIRR 0.95, 95% CI 0.92-0.97, p<0.001; overweight vs. normal weight aIRR 0.98, 95% CI 0.97-0.99, p=0.006; obese class I vs. normal weight aIRR 0.97, 95% CI 0.96-0.99, p<0.001) (Table 5). Obese class II/III had the largest impact on number of usable embryos (aIRR=0.95, 95% CI 0.93-0.97, p<0.001). When only blastocyst cycles were included, the difference in number of usable blastocysts was not statistically significant between overweight vs. normal weight patients, whereas the underweight and obese patients had fewer usable blastocysts than normal weight patients (Table 6).

TABLE 6
The effect of BMI on IVF success in blastocyst transfer cycles

| | | | | N (%) of cases | | aOR or aIRR | |
|---|---|---|---|---|---|---|---|
| | BMI (kg/m²) categories | | Total N | or mean ± SD | | (95% CI) | P value |
| Number of usable | Underweight | <18.5 | 934 | 4.1 ± 3.7 | | 0.95 (0.92, 0.98) | 0.002 |
| embryos | Normal | 18.5-24.9 | 19088 | 4.2 ± 3.8 | | 1.00 (reference) | |
| | Overweight | 25.0-29.9 | 8106 | 4.1 ± 3.7 | | 0.99 (0.97, 1.00) | 0.06 |
| | Obese class I | 30.0-34.9 | 3795 | 4.1 ± 3.7 | | 0.98 (0.96, 0.99) | 0.008 |
| | Obese class II/III | ≥35.0 | 2427 | 3.9 ± 3.8 | | 0.96 (0.94, 0.98) | <0.001 |
| Ongoing clinical | Underweight | <18.5 | 707 | 366 | (51.8) | 0.89 (0.76, 1.03) | 0.13 |
| pregnancy | Normal | 18.5-24.9 | 15259 | 8258 | (54.1) | 1.00 (reference) | |
| | Overweight | 25.0-29.9 | 6672 | 3458 | (51.8) | 0.94 (0.89, 1.00) | 0.05 |
| | Obese class I | 30.0-34.9 | 3180 | 1599 | (50.3) | 0.89 (0.82, 0.96) | 0.004 |
| | Obese class II/III | ≥35.0 | 2052 | 1005 | (49.0) | 0.85 (0.77, 0.93) | <0.001 |

For ongoing clinical pregnancy, factors that were controlled for in the analysis included: female age, basal E₂ levels, parity, infertility diagnosis, total gonadotropin used, number of oocytes retrieved, embryo stage at transfer, number of usable embryos, number of embryos transferred, and clinic. After adjusting for these confounders, among 39,055 patients with fresh embryo transfer, obese patients had lower odds of ongoing clinical pregnancy compared to normal weight patients (Table 5). When odds of ongoing clinical pregnancy were compared between obese class I and normal weight patients, aOR was 0.89 (95% CI 0.83-0.95, p<0.001), while aOR comparing obese class II/III and normal weight patients was 0.86 (95% CI 0.79-0.93, p<0.001). Underweight and overweight patients did not have significantly lower odds of ongoing clinical pregnancy compared to normal weight patients (p=0.21 and p=0.09 respectively). When only blastocyst transfers were included, results remained consistent, with obese patients having lower odds of ongoing clinical pregnancy (Table 6).

### EXAMPLE 4

In this example, a multi-center retrospective analysis and examined the effect of BMI on IVF cycle outcomes (cycle cancellation, oocytes retrieved, usable embryos, ongoing pregnancy) for 8 patient subpopulations with different primary infertility diagnosis, while controlling for confounding factors was performed.

### Study Design and Methodology

A retrospective review of 51,198 women who initiated their first autologous IVF or ICSI cycles at 13 fertility treatment centers in the United States between 2009-2015 was performed. Frozen transfers of supernumerary embryos, cycles using PGS, and cycles that were missing BMI data were excluded. BMI categories defined by WHO: <18.50 kg/m² (underweight), 18.50-24.99 kg/m² (normal), 25.00-29.99 kg/m² (overweight), 30.00-34.99 kg/m² (obese class I), 35.00-39.99 kg/m²(obese class II), and ≥40.00 kg/m² (obese class III) were used. Obese class II and class III were combined in our study because of limited sample size in obese class III group. The least absolute shrinkage and selection operator (LASSO) regression was used to selected confounders that were statistically significantly (p<0.05) associated with each outcome measure. For 8 patient subpopulations with different primary infertility diagnoses reported by the clinics (diminished ovarian reserve (DOR), endometriosis, idiopathic, male factor, ovulatory dysfunction, PCOS, tubal factor, and uterine factor), logistic or Poisson regression was used to calculate the multivariate aOR for cycle cancellation and ongoing clinical pregnancy or aIRR for number of oocytes retrieved and number of usable embryos), respectively, with their 95% CI, with normal BMI as the reference category.

### Results

We examined the effect of BMI on IVF cycle outcomes for patient subpopulations with different primary infertility diagnoses, and found that the effect of BMI on IVF cycle outcomes was more pronounced for PCOS, ovulatory dysfunction, male factor, and uterine factor patients (Tables 7-10).

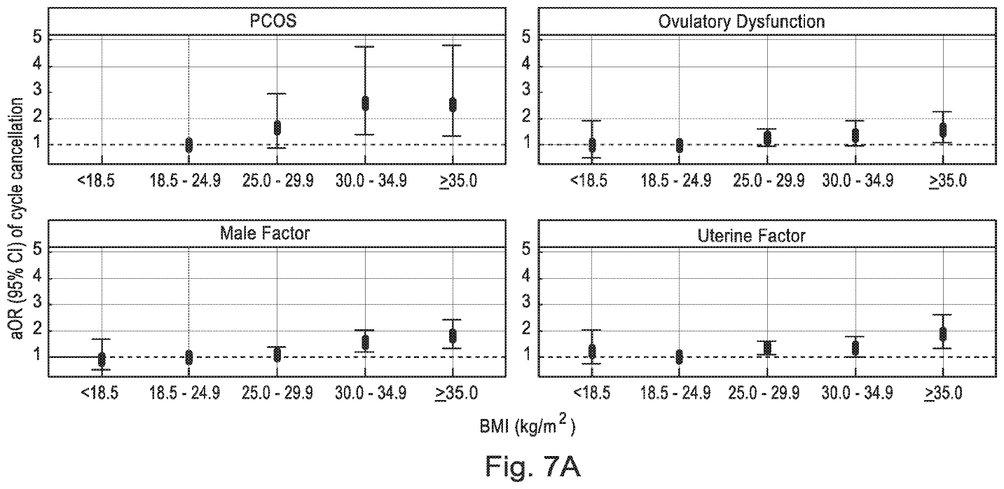

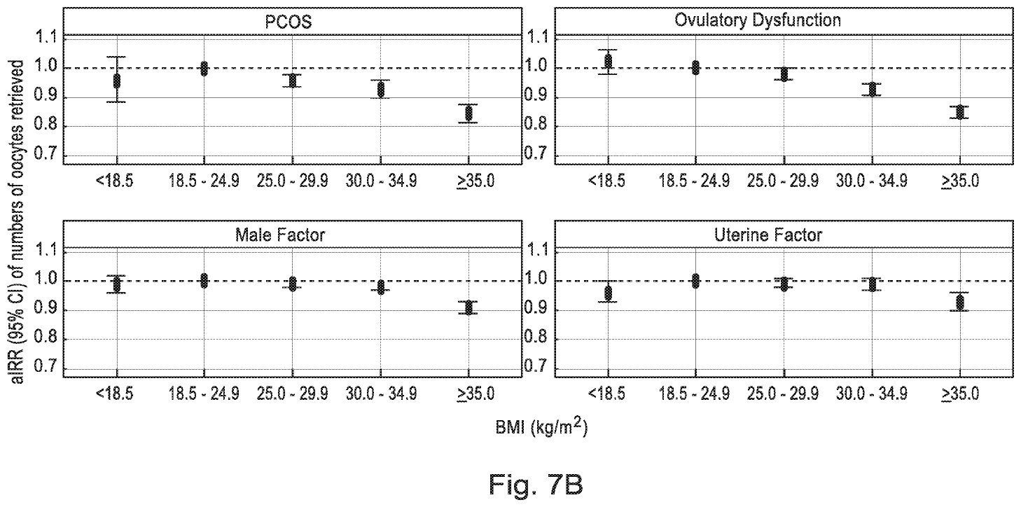

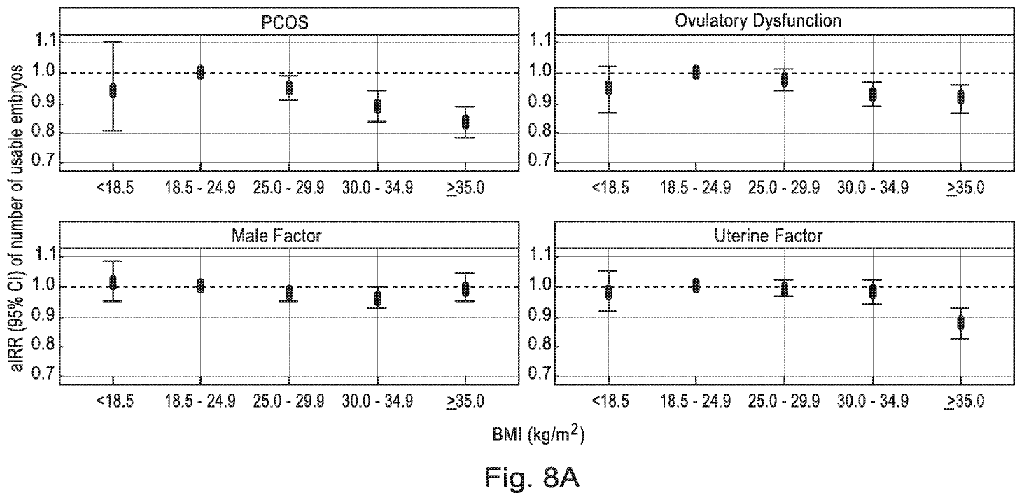

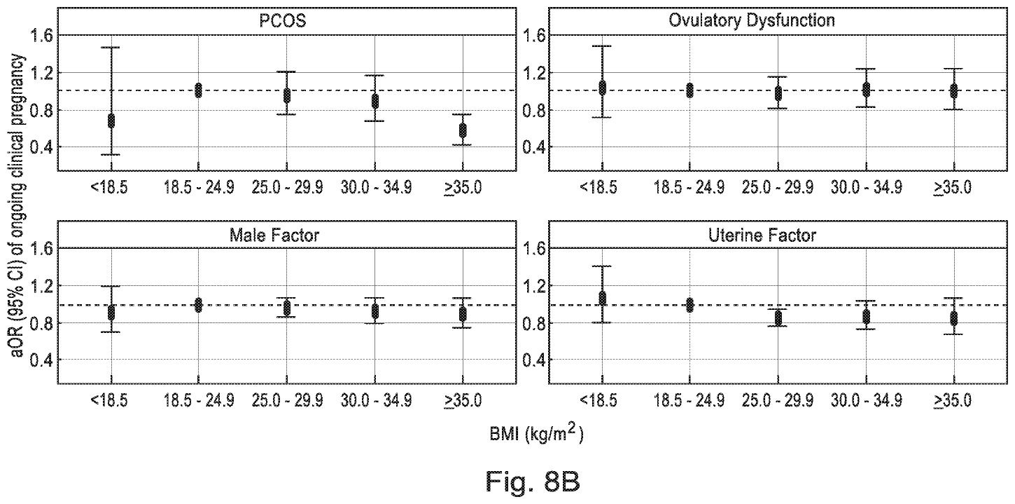

In cycles with primary diagnosis listed as “PCOS”, odds of cycle cancellation were higher in obese patients compared to normal weight patients (obese class I vs. normal weight: aOR 2.59, 95% CI 1.40-4.78, p=0.002; obese class II/III vs. normal weight: aOR 2.56, 95% CI 1.35-4.84, p=0.004) (FIG. 7A, Table 7). Both overweight and obese patients had fewer oocytes retrieved compared to normal weight patients (overweight vs. normal weight: aIRR 0.96, 95% CI 0.94-0.98, p=0.001; obese class I vs. normal weight: aIRR 0.93, 95% CI 0.90-0.96, p<0.001; obese class II/III vs. normal weight: aIRR 0.85, 95% CI 0.82-0.88, p<0.001) (FIG. 7B, Table 8). Overweight and obese patients also had fewer usable embryos compared to normal weight patients (overweight vs. normal weight: aIRR 0.95, 95% CI 0.91-0.99, p=0.03; obese class I vs. normal weight: aIRR 0.89, 95% CI 0.84-0.94, p<0.001; obese class II/III vs. normal weight: aIRR 0.84, 95% CI 0.79-0.89, p<0.001) (FIG. 8A, Table 9). For ongoing clinical pregnancy, only obese class II/III patients had poorer outcome than normal weight patients (aOR: 0.56, 95% CI 0.42-0.74, p<0.001) (FIG. 8B, Table 10).

In cycles with primary diagnosis listed as “Ovulatory dysfunction”, odds of cycle cancellation were higher in obese class II/III patients compared to normal weight patients (aOR 1.58, 95% CI 1.10-2.26, p=0.01) (FIG. 7A, Table 7). Both overweight and obese patients had fewer oocytes retrieved compared to normal weight patients (overweight vs. normal weight: aIRR 0.98, 95% CI 0.96-0.99, p=0.01; obese class I vs. normal weight: aIRR 0.93, 95% CI 0.91-0.95, p<0.001; obese class II/III vs. normal weight: aIRR 0.85, 95% CI 0.83-0.87, p<0.001) (FIG. 7B, Table 8). Obese patients also had fewer usable embryos compared to normal weight patients (obese class I vs. normal weight: aIRR 0.93, 95% CI 0.89-0.97, p=0.002; obese class II/III vs. normal weight: aIRR 0.92, 95% CI 0.87-0.96, p<0.001) (FIG. 8A, Table 9). For ongoing clinical pregnancy, underweight, overweight, and obese patients had similar outcome compared to normal weight patients (FIG. 8B, Table 10).

In cycles with primary diagnosis listed as “Male factor”, odds of cycle cancellation were higher in obese patients compared to normal weight patients (obese class I vs. normal weight: aOR 1.57, 95% CI 1.22-2.02, p<0.001; obese class II/III vs. normal weight: aOR 1.80, 95% CI 1.34-2.42, p<0.001) (FIG. 7A, Table 7). Obese class II/III patients had fewer oocytes retrieved compared to normal weight patients (aIRR 0.91, 95% CI 0.89-0.93, p<0.001) (FIG. 7B, Table 8), while obese class I patients had fewer usable embryos compared to normal weight patients (aIRR 0.96, 95% CI 0.93-0.99, p=0.04) (FIG. 8A, Table 9). For ongoing clinical pregnancy, underweight, overweight, and obese patients had similar outcome compared to normal weight patients (FIG. 8B, Table 10).

In cycles with primary diagnosis listed as “Uterine factor”, odds of cycle cancellation were higher in overweight and obese patients compared to normal weight patients (overweight vs. normal weight: aOR 1.33, 95% CI 1.09-1.61, p=0.004; obese class I vs. normal weight: aOR 1.35, 95% CI 1.03-1.77, p=0.03; obese class II/III vs. normal weight: aOR 1.90, 95% CI 1.37-2.63, p<0.001) (FIG. 7A, Table 7). Compared to normal weight patients, obese class II/III patients had fewer oocytes retrieved (aIRR 0.93, 95% CI 0.90-0.96, p<0.001) (FIG. 7B, Table 8), and fewer usable embryos (aIRR 0.88, 95% CI 0.83-0.93, p<0.001) (FIG. 8A, Table 9). Odds of ongoing clinical pregnancy of underweight, overweight, and obese patients were not statistically significantly different from their normal weight counterparts (FIG. 8B, Table 10).

TABLE 7
The effect of BMI on cycle cancellation among different infertility diagnosis

| | | | | N (%) of | | | |
|---|---|---|---|---|---|---|---|
| Diagnosis | BMI (kg/m²) categories | | Total (N) | cases | | aOR (95% CI) | P value |
| DOR | Underweight | <18.5 | 202 | 40 | (19.8) | 1.09 (0.74, 1.60) | 0.66 |
| | Normal | 18.5-24.9 | 4514 | 947 | (21.0) | 1.00 (reference) | |
| | Overweight | 25.0-29.9 | 1901 | 427 | (22.5) | 1.10 (0.95, 1.27) | 0.19 |
| | Obese class I | 30.0-34.9 | 776 | 174 | (22.4) | 1.11 (0.90, 1.36) | 0.33 |
| | Obese class II/III | ≥35.0 | 488 | 121 | (24.8) | 1.17 (0.92, 1.49) | 0.2 |
| Endometriosis | Underweight | <18.5 | 102 | 9 | (8.8) | 1.14 (0.52, 2.51) | 0.74 |
| | Normal | 18.5-24.9 | 1707 | 131 | (7.7) | 1.00 (reference) | |
| | Overweight | 25.0-29.9 | 620 | 75 | (12.1) | 1.44 (1.03,2.01) | 0.04 |
| | Obese class I | 30.0-34.9 | 241 | 26 | (10.8) | 1.31 (0.80, 2.16) | 0.28 |
| | Obese class II/III | ≥35.0 | 87 | 7 | (8.0) | 0.91 (0.38, 2.15) | 0.82 |
| Idiopathic | Underweight | <18.5 | 57 | 6 | (10.5) | 3.74 (1.40,9.95) | 0.008 |
| | Normal | 18.5-24.9 | 1192 | 74 | (6.2) | 1.00 (reference) | |
| | Overweight | 25.0-29.9 | 373 | 39 | (10.5) | 1.44 (0.90, 2.32) | 0.13 |
| | Obese class I | 30.0-34.9 | 145 | 9 | (6.2) | 0.63 (0.28, 1.44) | 0.27 |
| | Obese class II/III | ≥35.0 | 86 | 6 | (7.0) | 1.12 (0.44, 2.84) | 0.82 |
| Male factor | Underweight | <18.5 | 282 | 12 | (4.3) | 0.92 (0.50, 1.71) | 0.8 |
| | Normal | 18.5-24.9 | 5583 | 314 | (5.6) | 1.00 (reference) | |
| | Overweight | 25.0-29.9 | 2552 | 173 | (6.8) | 1.15 (0.94, 1.41) | 0.18 |
| | Obese class I | 30.0-34.9 | 1167 | 100 | (8.6) | 1.57 (1.22, 2.02) | <0.001 |
| | Obese class II/III | ≥35.0 | 742 | 70 | (9.4) | 1.80 (1.34, 2.42) | <0.001 |
| Ovulatory | Underweight | <18.5 | 168 | 12 | (7.1) | 1.02 (0.54, 1.96) | 0.94 |
| dysfunction | Normal | 18.5-24.9 | 2560 | 182 | (7.1) | 1.00 (reference) | |
| | Overweight | 25.0-29.9 | 1094 | 97 | (8.9) | 1.25 (0.95, 1.66) | 0.12 |
| | Obese class I | 30.0-34.9 | 657 | 58 | (8.8) | 1.37 (0.97, 1.92) | 0.07 |
| | Obese class II/III | ≥35.0 | 584 | 50 | (8.6) | 1.58 (1.10, 2.26) | 0.01 |
| PCOS | Underweight | <18.5 | 33 | 0 | (0) | 0 (0, Inf) | 0.99 |
| | Normal | 18.5-24.9 | 940 | 26 | (2.8) | 1.00 (reference) | |
| | Overweight | 25.0-29.9 | 552 | 22 | (4.0) | 1.64 (0.91,2.97) | 0.1 |
| | Obese class I | 30.0-34.9 | 383 | 22 | (5.7) | 2.59 (1.40, 4.78) | 0.002 |
| | Obese class II/III | ≥35.0 | 405 | 24 | (5.9) | 2.56 (1.35,4.84) | 0.004 |
| Tubal factor | Underweight | <18.5 | 58 | 9 | (15.5) | 2.09 (0.91, 4.82) | 0.08 |
| | Normal | 18.5-24.9 | 1247 | 119 | (9.5) | 1.00 (reference) | |
| | Overweight | 25.0-29.9 | 645 | 63 | (9.8) | 0.94 (0.65, 1.36) | 0.75 |
| | Obese class I | 30.0-34.9 | 327 | 36 | (11.0) | 1.4 (0.89, 2.21) | 0.15 |
| | Obese class II/III | ≥35.0 | 188 | 19 | (10.1) | 1.36 (0.76, 2.46) | 0.3 |
| Uterine factor | Underweight | <18.5 | 271 | 22 | (8.1) | 1.26 (0.77, 2.04) | 0.36 |
| | Normal | 18.5-24.9 | 5480 | 429 | (7.8) | 1.00 (reference) | |
| | Overweight | 25.0-29.9 | 2010 | 201 | (10.0) | 1.33 (1.09, 1.61) | 0.004 |
| | Obese class I | 30.0-34.9 | 812 | 90 | (11.1) | 1.35 (1.03, 1.77) | 0.03 |
| | Obese class II/III | ≥35.0 | 463 | 62 | (13.4) | 1.90 (1.37, 2.63) | <0.001 |

Note:
aOR and 95% CI were calculated after adjustment for female age, BAFC, day 3 E₂, day 3 FSH, AMH, gravidity, clinic, and total gonadotropin dose.

TABLE 8
The effect of BMI on number of oocytes retrieved among different infertility diagnosis

| Diagnosis | BMI (kg/m²) categories | | Total (N) | Mean ± SD | aIRR (95% CI) | P value |
|---|---|---|---|---|---|---|
| DOR | Underweight | <18.5 | 162 | 7.9 ± 5.8 | 0.93 (0.88, 0.98) | 0.01 |
| | Normal | 18.5-24.9 | 3567 | 8.4 ± 5.8 | 1.00 (reference) | |
| | Overweight | 25.0-29.9 | 1474 | 8.7 ± 6.0 | 1.02(1.00, 1.04) | 0.06 |
| | Obese class I | 30.0-34.9 | 602 | 8.9 ± 6.4 | 1.03 (1.00, 1.06) | 0.05 |
| | Obese class II/III | ≥35.0 | 367 | 8.5 ± 5.6 | 1.00 (0.96, 1.04) | 0.99 |
| Endometriosis | Underweight | <18.5 | 93 | 13.2 ± 8.8 | 0.98 (0.93, 1.04) | 0.59 |
| | Normal | 18.5-24.9 | 1576 | 13.3 ± 7.5 | 1.00 (reference) | |
| | Overweight | 25.0-29.9 | 545 | 13.0 ± 7.7 | 1.01 (0.98, 1.04) | 0.39 |
| | Obese class I | 30.0-34.9 | 215 | 12.7 ± 7.5 | 0.99 (0.95, 1.03) | 0.7 |
| | Obese class II/III | ≥35.0 | 80 | 11.6 ± 7.0 | 0.99 (0.93, 1.06) | 0.81 |
| Idiopathic | Underweight | <18.5 | 51 | 14.9 ± 8.2 | 0.99 (0.92, 1.06) | 0.73 |
| | Normal | 18.5-24.9 | 1118 | 14.3 ± 8.1 | 1.00 (reference) | |
| | Overweight | 25.0-29.9 | 334 | 15.1 ± 8.7 | 1.07(1.04, 1.11) | <0.001 |
| | Obese class I | 30.0-34.9 | 136 | 13.7 ± 7.8 | 1.01 (0.97, 1.07) | 0.55 |
| | Obese class II/III | ≥35.0 | 80 | 13.4 ± 7.1 | 1.01 (0.95, 1.08) | 0.74 |
| Male factor | Underweight | <18.5 | 270 | 16.1 ± 9.4 | 0.99 (0.96, 1.02) | 0.41 |
| | Normal | 18.5-24.9 | 5269 | 15.4 ± 8.3 | 1.00 (reference) | |
| | Overweight | 25.0-29.9 | 2379 | 15.0 ± 8.2 | 0.99 (0.98, 1.00) | 0.24 |
| | Obese class I | 30.0-34.9 | 1067 | 14.7 ± 8.4 | 0.98 (0.97, 1.00) | 0.06 |
| | Obese class II/III | ≥35.0 | 672 | 13.3 ± 7.5 | 0.91 (0.89, 0.93) | <0.001 |
| Ovulatory | Underweight | <18.5 | 156 | 17.0 ± 10.0 | 1.02 (0.98, 1.06) | 0.29 |
| dysfunction | Normal | 18.5-24.9 | 2378 | 16.8 ± 9.5 | 1.00 (reference) | |
| | Overweight | 25.0-29.9 | 997 | 16.6 ± 9.8 | 0.98 (0.96, 0.99) | 0.01 |
| | Obese class I | 30.0-34.9 | 599 | 15.9 ± 9.3 | 0.93 (0.91, 0.95) | <0.001 |
| | Obese class II/III | ≥35.0 | 534 | 14.8 ± 8.5 | 0.85 (0.83, 0.87) | <0.001 |
| PCOS | Underweight | <18.5 | 33 | 20.5 ± 7.6 | 0.96 (0.89, 1.04) | 0.28 |
| | Normal | 18.5-24.9 | 914 | 20.6 ± 10.3 | 1.00 (reference) | |
| | Overweight | 25.0-29.9 | 530 | 19.6 ± 10.4 | 0.96 (0.94, 0.98) | 0.001 |
| | Obese class I | 30.0-34.9 | 361 | 18.8 ± 10.8 | 0.93 (0.90, 0.96) | <0.001 |
| | Obese class II/III | ≥35.0 | 381 | 16.1 ± 9.0 | 0.85 (0.82, 0.88) | <0.001 |
| Tubal factor | Underweight | <18.5 | 49 | 17.3 ± 10.6 | 1.11 (1.04, 1.19) | 0.003 |
| | Normal | 18.5-24.9 | 1128 | 14.5 ± 8.8 | 1.00 (reference) | |
| | Overweight | 25.0-29.9 | 582 | 14.4 ± 9.0 | 1.01 (0.98, 1.03) | 0.64 |
| | Obese class I | 30.0-34.9 | 291 | 14.5 ± 9.4 | 1.01 (0.97, 1.04) | 0.71 |
| | Obese class II/III | ≥35.0 | 169 | 13.5 ± 7.5 | 0.96 (0.92, 1.00) | 0.07 |
| Uterine factor | Underweight | <18.5 | 249 | 13.8 ± 7.6 | 0.96 (0.93, 0.99) | 0.03 |
| | Normal | 18.5-24.9 | 5051 | 14.3 ± 7.8 | 1.00 (reference) | |
| | Overweight | 25.0-29.9 | 1809 | 13.9 ± 7.6 | 0.99 (0.98, 1.01) | 0.25 |
| | Obese class I | 30.0-34.9 | 722 | 13.9 ± 7.8 | 0.99 (0.97, 1.01) | 0.42 |
| | Obese class II/III | ≥35.0 | 401 | 12.5 ± 7.1 | 0.93 (0.90, 0.96) | <0.001 |

Note:
aIRR and 95% CI were calculated after adjustment for female age, BAFC, day 3 E₂, day 3 LH, day 3 FSH, AMH, parity, clinic, and total gonadotropin dose.

TABLE 9
The effect of BMI on number of usable embryos among different infertility diagnosis

| Diagnosis | BMI (kg/m²) categories | | Total (N) | Mean ± SD | aIRR (95% CI) | P value |
|---|---|---|---|---|---|---|
| DOR | Underweight | <18.5 | 161 | 2.1 ± 1.9 | 0.92 (0.83, 1.03) | 0.15 |
| | Normal | 18.5-24.9 | 3523 | 2.3 ± 1.9 | 1.00 (reference) | |
| | Overweight | 25.0-29.9 | 1454 | 2.4 ± 1.9 | 1.01 (0.97, 1.05) | 0.79 |
| | Obese class I | 30.0-34.9 | 596 | 2.5 ± 1.8 | 1.01 (0.96, 1.07) | 0.69 |
| | Obese class II/III | ≥35.0 | 358 | 2.4 ± 1.7 | 1.06 (0.99, 1.14) | 0.11 |
| Endometriosis | Underweight | <18.5 | 92 | 3.4 ± 2.8 | 0.92 (0.82, 1.03) | 0.15 |
| | Normal | 18.5-24.9 | 1573 | 3.6 ± 2.9 | 1.00 (reference) | |
| | Overweight | 25.0-29.9 | 543 | 3.7 ± 3.4 | 1.00 (0.95, 1.06) | 0.87 |
| | Obese class I | 30.0-34.9 | 215 | 3.8 ± 3.4 | 1.07 (0.99, 1.15) | 0.07 |
| | Obese class II/III | ≥35.0 | 80 | 3.7 ± 3.1 | 1.08 (0.96, 1.21) | 0.23 |
| Idiopathic | Underweight | <18.5 | 51 | 4.1 ± 3.7 | 1.08 (0.94, 1.24) | 0.28 |
| | Normal | 18.5-24.9 | 1111 | 4.0 ± 4.7 | 1.00 (reference) | |
| | Overweight | 25.0-29.9 | 333 | 4.4 ± 4.7 | 1.03 (0.97, 1.10) | 0.29 |
| | Obese class I | 30.0-34.9 | 135 | 3.3 ± 3.0 | 0.86 (0.78, 0.95) | 0.003 |
| | Obese class II/III | ≥35.0 | 79 | 3.3 ± 2.3 | 0.89 (0.78, 1.01) | 0.06 |
| Male factor | Underweight | <18.5 | 270 | 4.0 ± 3.8 | 1.01 (0.95, 1.08) | 0.71 |
| | Normal | 18.5-24.9 | 5263 | 3.7 ± 2.9 | 1.00 (reference) | |
| | Overweight | 25.0-29.9 | 2376 | 3.5 ± 2.8 | 0.98 (0.95, 1.00) | 0.06 |
| | Obese class I | 30.0-34.9 | 1063 | 3.4 ± 2.8 | 0.96 (0.93, 0.99) | 0.04 |
| | Obese class II/III | ≥35.0 | 669 | 3.3 ± 2.5 | 0.99 (0.95, 1.04) | 0.75 |
| Ovulatory | Underweight | <18.5 | 156 | 4.3 ± 3.4 | 0.95 (0.87, 1.02) | 0.17 |
| dysfunction | Normal | 18.5-24.9 | 2374 | 4.5 ± 3.8 | 1.00 (reference) | |
| | Overweight | 25.0-29.9 | 996 | 4.3 ± 3.8 | 0.98 (0.94, 1.01) | 0.18 |
| | Obese class I | 30.0-34.9 | 597 | 3.9 ± 3.2 | 0.93 (0.89, 0.97) | 0.002 |
| | Obese class II/III | ≥35.0 | 530 | 3.7 ± 2.8 | 0.92 (0.87, 0.96) | <0.001 |
| PCOS | Underweight | <18.5 | 33 | 5.3 ± 4.2 | 0.94 (0.81, 1.10) | 0.43 |
| | Normal | 18.5-24.9 | 912 | 5.9 ± 5.0 | 1.00 (reference) | |
| | Overweight | 25.0-29.9 | 529 | 5.4 ± 4.5 | 0.95 (0.91, 0.99) | 0.03 |
| | Obese class I | 30.0-34.9 | 359 | 4.9 ± 4.3 | 0.89 (0.84, 0.94) | <0.001 |
| | Obese class II/III | ≥35.0 | 380 | 4.0 ± 3.3 | 0.84 (0.79, 0.89) | <0.001 |
| Tubal factor | Underweight | <18.5 | 49 | 3.1 ± 2.8 | 0.68 (0.58, 0.80) | <0.001 |
| | Normal | 18.5-24.9 | 1123 | 3.9 ± 4.0 | 1.00 (reference) | |
| | Overweight | 25.0-29.9 | 580 | 3.7 ± 3.9 | 0.92 (0.87, 0.97) | 0.003 |
| | Obese class I | 30.0-34.9 | 290 | 4.1 ± 4.5 | 0.96 (0.90, 1.03) | 0.25 |
| | Obese class II/III | ≥35.0 | 169 | 3.5 ± 3.3 | 0.90 (0.82, 0.98) | 0.02 |
| Uterine factor | Underweight | <18.5 | 248 | 3.7 ± 2.8 | 0.98 (0.92, 1.05) | 0.62 |
| | Normal | 18.5-24.9 | 5044 | 3.8 ± 3.0 | 1.00 (reference) | |
| | Overweight | 25.0-29.9 | 1806 | 3.6 ± 2.8 | 0.99 (0.97, 1.02) | 0.62 |
| | Obese class I | 30.0-34.9 | 721 | 3.5 ± 2.8 | 0.98 (0.94, 1.02) | 0.38 |
| | Obese class II/III | ≥35.0 | 398 | 2.9 ± 2.2 | 0.88 (0.83, 0.93) | <0.001 |

Note:
aIRR and 95% CI were calculated after adjustment for female age, BAFC, day 3 E₂, parity, embryo stage, clinic, total gonadotropin dose, and number of oocytes retrieved.

TABLE 10
The effect of BMI on ongoing clinical pregnancy among different infertility diagnosis

| | | | | N (%) of | | | |
|---|---|---|---|---|---|---|---|
| Diagnosis | BMI (kg/m²) categories | | Total (N) | cases | | aOR (95% CI) | P value |
| DOR | Underweight | <18.5 | 113 | 32 | (28.3) | 0.87 (0.56, 1.36) | 0.55 |
| | Normal | 18.5-24.9 | 2734 | 842 | (30.8) | 1.00 (reference) | |
| | Overweight | 25.0-29.9 | 1145 | 357 | (31.2) | 1.12(0.96, 1.32) | 0.16 |
| | Obese class I | 30.0-34.9 | 501 | 121 | (24.2) | 0.76 (0.60, 0.96) | 0.02 |
| | Obese class II/III | ≥35.0 | 301 | 88 | (29.2) | 1.10(0.83, 1.46) | 0.5 |
| Endometriosis | Underweight | <18.5 | 76 | 33 | (43.4) | 0.80 (0.49, 1.31) | 0.38 |
| | Normal | 18.5-24.9 | 1431 | 713 | (49.8) | 1.00 (reference) | |
| | Overweight | 25.0-29.9 | 488 | 222 | (45.5) | 0.88 (0.71, 1.10) | 0.26 |
| | Obese class I | 30.0-34.9 | 198 | 82 | (41.4) | 0.73 (0.53, 1.00) | 0.05 |
| | Obese class II/III | ≥35.0 | 73 | 28 | (38.4) | 0.73 (0.44, 1.22) | 0.23 |
| Idiopathic | Underweight | <18.5 | 46 | 16 | (34.8) | 0.67 (0.35, 1.27) | 0.22 |
| | Normal | 18.5-24.9 | 940 | 429 | (45.6) | 1.00 (reference) | |
| | Overweight | 25.0-29.9 | 280 | 125 | (44.6) | 1.01 (0.76, 1.35) | 0.92 |
| | Obese class I | 30.0-34.9 | 107 | 46 | (43) | 1.02 (0.67, 1.56) | 0.92 |
| | Obese class II/III | ≥35.0 | 68 | 23 | (33.8) | 0.71 (0.41, 1.23) | 0.22 |
| Male factor | Underweight | <18.5 | 230 | 113 | (49.1) | 0.92 (0.70, 1.21) | 0.53 |
| | Normal | 18.5-24.9 | 4636 | 2322 | (50.1) | 1.00 (reference) | |
| | Overweight | 25.0-29.9 | 2132 | 1026 | (48.1) | 0.96 (0.87, 1.07) | 0.51 |
| | Obese class I | 30.0-34.9 | 953 | 450 | (47.2) | 0.93 (0.80, 1.07) | 0.32 |
| | Obese class II/III | ≥35.0 | 612 | 280 | (45.8) | 0.9 (0.75, 1.07) | 0.23 |
| Ovulatory | Underweight | <18.5 | 126 | 67 | (53.2) | 1.03 (0.71, 1.49) | 0.89 |
| dysfunction | Normal | 18.5-24.9 | 1898 | 1004 | (52.9) | 1.00 (reference) | |
| | Overweight | 25.0-29.9 | 840 | 424 | (50.5) | 0.96 (0.81, 1.14) | 0.64 |
| | Obese class I | 30.0-34.9 | 517 | 261 | (50.5) | 1.01 (0.82, 1.24) | 0.96 |
| | Obese class II/III | ≥35.0 | 464 | 233 | (50.2) | 0.99 (0.80, 1.24) | 0.96 |
| PCOS | Underweight | <18.5 | 27 | 13 | (48.1) | 0.66 (0.30, 1.47) | 0.31 |
| | Normal | 18.5-24.9 | 748 | 456 | (61.0) | 1.00 (reference) | |
| | Overweight | 25.0-29.9 | 470 | 275 | (58.5) | 0.95 (0.74, 1.21) | 0.66 |
| | Obese class I | 30.0-34.9 | 312 | 176 | (56.4) | 0.88 (0.66, 1.16) | 0.36 |
| | Obese class II/III | ≥35.0 | 340 | 143 | (42.1) | 0.56 (0.42, 0.74) | <0.001 |
| Tubal factor | Underweight | <18.5 | 36 | 16 | (44.4) | 0.92 (0.46, 1.85) | 0.82 |
| | Normal | 18.5-24.9 | 864 | 404 | (46.8) | 1.00 (reference) | |
| | Overweight | 25.0-29.9 | 460 | 195 | (42.4) | 0.98 (0.77, 1.24) | 0.84 |
| | Obese class I | 30.0-34.9 | 235 | 95 | (40.4) | 0.78 (0.57, 1.07) | 0.12 |
| | Obese class II/III | ≥35.0 | 140 | 59 | (42.1) | 0.89 (0.61, 1.31) | 0.55 |
| Uterine factor | Underweight | <18.5 | 215 | 111 | (51.6) | 1.07 (0.81, 1.43) | 0.63 |
| | Normal | 18.5-24.9 | 4519 | 2231 | (49.4) | 1.00 (reference) | |
| | Overweight | 25.0-29.9 | 1656 | 733 | (44.3) | 0.85 (0.76, 0.96) | 0.008 |
| | Obese class I | 30.0-34.9 | 662 | 289 | (43.7) | 0.87 (0.73, 1.04) | 0.12 |
| | Obese class II/III | ≥35.0 | 360 | 146 | (40.6) | 0.85 (0.68, 1.07) | 0.16 |

Note:
aOR and 95% CI were calculated after adjustment for female age, day 3 E₂, parity, embryo stage, clinic, total gonadotropin dose, number of oocytes retrieved, number of usable embryos, number of embryos transferred.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore

1-30. (canceled)

31. A method for analyzing a patient's potential for achieving ongoing pregnancy with respect to a specific fertility treatment comprising:
a) measuring a plurality of clinical characteristics in a patient, the characteristics selected from the group consisting of: age, weight, basal antral follicle count (BAFC), and current medications;
b) determining body mass index (BMI) of said patient;
c) comparing results obtained from said measuring and determining steps to reference data comprising BMI and at least one of: age, weight, BAFC, and medications taken, said reference data being obtained from female patients whose treatment and fertility outcome are known; and
d) identifying a course of treatment for said specific fertility treatment for said patient based on a best fit between said clinical characteristics and said body mass index BMI of said patient and said reference data.

32. The method of claim 31, further comprising predicting a probability of achieving ongoing pregnancy using said course of treatment for said specific fertility treatment for said patient.

33. The method of claim 31, further comprising administering said course of treatment for said specific fertility treatment to said patient.

34. The method of claim 31, wherein said course of treatment comprises a treatment is selected from the group consisting of: intrauterine insemination (IUI), ovulation induction, IUI with ovulation induction, in vitro fertilization (IVF), and IVF with intra-cytoplasmic sperm injection (ICSI).

35. The method of claim 34, wherein the course of treatment comprises the administration of one or more ovulation induction agents.

36. The method of claim 35, wherein the one or more ovulation induction agents comprise a gonadotropin.

37. The method of claim 36, wherein the gonadotropin comprises luteinizing hormone (LH), follicle stimulating hormone (FSH), or both LH and FSH.

38. The method of claim 36, wherein the gonadotropin comprises human chorionic gonadotropin (hCG).

39. The method of claim 35, wherein the one or more ovulation induction agents comprise an oral ovulation induction agent.

40. The method of claim 39, wherein the oral ovulation induction agent is selected from the group consisting of: clomiphene citrate, letrozole, anastrozole, metformin, rosiglitazone, pioglitazone, bromocriptine, cabergoline, gonadotropin-releasing hormone (GnRH), leuprolide acetate, nafarelin acetate, goserelin acetate, ganirelix, and cetrorelix acetate.

41. The method of claim 31, wherein the plurality of clinical characteristics further comprise a level of a fertility-related hormone in the patient.

42. The method of claim 34, wherein the course of treatment comprises administering:
a) a gonadotropin alone if the patient's BMI is above a threshold limit; or
b) a gonadotropin and an oral ovulation induction agent if the patient's BMI is below the threshold limit.

43. The method of claim 42, wherein the threshold limit is about 20, 25, or 30.

44. The method of claim 42, further comprising conducting an assay to measure a level of one or more fertility-related hormones in the patient and determining which gonadotropin to administer based at least on the level of the one or more fertility-related hormones.

45. The method of claim 31, further comprising conducting an assay on a sample obtained from the patient to determine one or more genetic characteristics of the patient.

46. The method of claim 45, wherein the reference data further comprises genetic characteristics.

47. The method of claim 46, wherein the comparing step further comprises comparing results obtained from the conducting an assay step to the reference set of data.

48. The method of claim 45, wherein the one or more genetic characteristics of the patient comprises a genetic variant in, or abnormal expression of, one or more genes listed in Table 3.

49. The method of claim 31, wherein said comparing step is performed using a computer system comprising at least one processor coupled to a non-transitory memory storing said reference data.

50. A computer-implemented system comprising: at least one processor, a memory, and a computer program including instructions executable by the at least one processor to create an application for analyzing a patient's potential for achieving ongoing pregnancy with respect to a specific fertility treatment, the application comprising:
a) a software module receiving a plurality of clinical characteristics of a patient, the characteristics selected from the group consisting of: age, weight, basal antral follicle count (BAFC), and current medications;
b) a software module receiving a body mass index (BMI) of said patient;
c) a software module comparing said plurality of clinical characteristics and said BMI to reference data comprising BMI and at least one of: age, weight, BAFC, and medications taken, said reference data being obtained from female patients whose treatment and fertility outcome are known; and
d) identifying a course of treatment for said specific fertility treatment for said patient based on a best fit between said clinical characteristics and said BMI of said patient and said reference data.
